(19) United States
(12) Patent Application Publication
(10) Pub. No.: US 2024/0366507 A1
(43) Pub. Date: Nov. 7, 2024

(54) Methods of Treating Neurologic Disorders

(71) Applicants: SYNERGENE THERAPEUTICS, INC., Potomac, MD (US); GEORGETOWN UNIVERSITY, Washington, DC (US)

(72) Inventors: Esther H. CHANG, Potomac, MD (US); Joe B. HARFORD, Potomac, MD (US)

(21) Appl. No.: 18/612,306

(22) Filed: Mar. 21, 2024

Related U.S. Application Data
(60) Provisional application No. 63/493,197, filed on Mar. 30, 2023.

(51) Int. Cl. A61K 9/127; A61K 9/00; A61K 39/00; C07K 16/28

(52) U.S. Cl. CPC A61K 9/127; A61K 9/0019; C07K 16/2881; A61K 2039/505; C07K 2317/622

(57) ABSTRACT

This invention provides methods of treating neurologic disorders including, but not limited to, synucleinopathic disorders (including Parkinson's Disease), Alzheimer's Disease, Amyotrophic Lateral Sclerosis (ALS) disease and Huntington's disease. The methods include delivery into neuronal cells of antibody or antibody fragment(s), or nucleic acids encoding intrabodies, such as single domain antibodies and single chain Fv antibody fragments against proteins associated with such neurologic disorders, suitably using a liposomal complex that crosses the blood brain barrier and can deliver the payload into neuronal cells. This invention also provides methods wherein a delivered nucleic acid encodes a protein which is a nanobody targeted to a neuronal postsynaptic scaffolding protein Homer1 that is present at partially overlapping sets of excitatory synapses, and is associated with Fragile X Syndrome (FXS) and Deafness, Autosomal Dominant 68 (DFNA68).

### CROSS-REFERENCE TO RELATED APPLICATIONS

The present application claims benefit of U.S. Provisional Application No. 63/493,197, filed Mar. 30, 2023, the disclosure of which is incorporated by reference herein in its entirety.

### FIELD OF THE INVENTION

This invention provides methods of treating neurologic disorders including, but not limited to, synucleinopathic disorders (including Parkinson's Disease), Alzheimer's Disease, Amyotrophic Lateral Sclerosis (ALS) disease and Huntington's disease. The methods include delivery into neuronal cells of antibody or antibody fragment(s), or nucleic acids encoding intrabodies, such as single domain antibodies and single chain Fv antibody fragments against proteins associated with such neurologic disorders, suitably using a liposomal complex that crosses the blood brain barrier and can deliver the payload into neuronal cells. This invention also provides methods wherein a delivered nucleic acid encodes a protein which is a nanobody targeted to a neuronal postsynaptic scaffolding protein Homer1 that is present at partially overlapping sets of excitatory synapses, and is associated with Fragile X Syndrome (FXS) and Deafness, Autosomal Dominant 68 (DFNA68).

### BACKGROUND OF THE INVENTION

Antibodies of all types (murine, chimeric, humanized and human) have been approved by Food and Drug Administration (FDA), European Medicines Agency (EMA) and other national agencies for the treatment of several diseases, including neurological diseases. Many of the mAbs used in neurology today have been repurposed from their original indications for hematological neoplasias (e.g., alemtuzumab, ofatumumab and rituximab) or rheumatological disease (e.g., tocilizumab) [Gklinos, P.; Papadopoulou, M.; Stanulovic, V.; Mitsikostas, D. D.; Papadopoulos, D. Monoclonal Antibodies as Neurological Therapeutics. Pharmaceuticals 2021, 14, 92. https://doi.org/10.3390/ph1402 0092]. Other mAbs have been developed originally for neurological disease (e.g., ocrelizumab for multiple sclerosis or mAbs for migraine prophylaxis). Sixteen marketed mAbs are used in neurology primarily for neuroimmunological conditions and migraine and additional mAbs are in development for neurodegenerative conditions.

A notable drawback of using mAbs for neurologic diseases is their low accessibility to the CNS compartment. The normal brain-to-blood IgG concentration ratio of IV infused mAbs is approximately 0.1%. A recent double-blind trial investigated the effects of intrathecal and intravenous administration of rituximab versus placebo on a number of biomarkers of B cells depletion, inflammation and neurodegeneration in progressive Multiple Sclerosis (MS) (RIVITALISE trial; NCT01212094). The trial was discontinued early because at interim analysis, cerebrospinal fluid (CSF) B cells were only partially and transiently depleted and neurofilament light chain levels used as a marker of axonal damage were unchanged. The study identified low CSF levels of lytic complement factors and paucity of cytotoxic CD56dim NK cells as key contributors to decreased efficacy of intrathecally-administered rituximab [Komori, M.; Lin, Y. C.; Cortese, I.; Blake, A.; Ohayon, J.; Cherup, J.; Maric, D.; Kosa, P. T.; Wu, T.; Bielekova, B. Insufficient disease inhibition by intrathecal rituximab in progressive multiple sclerosis. Ann. Clin. Transl. Neurol. 2016, 3, 166-179.].

There are major limitations to current approaches for the intracellular delivery of antibodies. Direct physical delivery of the antibody or intrabody gene for expression is not feasible for clinical translation and also has safety and efficiency issues. Fusion of the antibodies with internalizing antibodies or protein-transduction domains have questionable efficiency, a lack of tissue and cellular specificity and there are issues with the efficiency of endosomal escape. The majority of nanocarriers also have problems with cellular specificity, endosomal escape and, depending on their size, there may also be immunogenicity issues [Slastnikova T A, Ulasov A V, Rosenkranz A A, Sobolev A S. Targeted Intracellular Delivery of Antibodies: The State of the Art. Front Pharmacol. 2018 Oct. 24; 9:1208. doi: 10.3389/fphar.2018.01208. PMID: 30405420; PMCID: PMC6207587]. What is needed is a method for treatment of neurologic disorders that overcomes the problems with delivering antibodies and genes encoding antibodies across the blood-brain barrier and into neuronal cells. The present invention fulfils these needs.

### BRIEF SUMMARY OF THE INVENTION

In embodiments, provided herein is a method of treating a neurologic disorder in a patient, comprising: providing a transferrin receptor-targeted cationic liposomal complex, comprising, a cationic liposome, a transferrin receptor-targeting moiety that is complexed with the cationic liposome, but is not chemically conjugated to the cationic liposome, and wherein the targeting moiety does not comprise a lipid tag, and a nucleic acid encoding an intrabody against a protein associated with a neurologic disorder and administering the transferrin receptor-targeted cationic liposomal complex to the patient to treat the disorder. Also, in suitable embodiments the gene encodes a protein which is a nanobody targeted to a neuronal postsynaptic scaffolding protein, Homer1, that is present at partially overlapping sets of excitatory synapses, and is associated with Fragile X Syndrome (FXS) and Deafness Autosomal Dominant 68 (DFNA68). In other embodiments, the transferrin receptor-targeted cationic liposomal complex encapsulates and delivers an antibody/antibody fragment against a protein associated with a neurologic disorder and administering the transferrin receptor-targeted cationic liposomal complex to the patient to treat the disorder.

### BRIEF DESCRIPTION OF DRAWINGS

The foregoing and other features and aspects of the present technology can be better understood from the following description of embodiments and as illustrated in the accompanying drawings. The accompanying drawings, which are incorporated herein and form a part of the specification, further serve to illustrate the principles of the present technology. The components in the drawings are not necessarily to scale.

FIG. 1 shows the method of treatment approach described herein.

FIG. 2 shows further details regarding the method of treatment approach described herein.

FIG. 3 shows the ability of the nanocomplexes comprising TfRscFv as the targeting moiety described herein to cross the blood-brain barrier.

FIG. 4 shows in vivo neuronal transfections of hSlo-EGFP cDNA using the nanocomplexes comprising Tf as the targeting moiety, as described herein.

### DETAILED DESCRIPTION OF THE INVENTION

Ever-increasing use of mAbs has been associated with several immune-mediated and other adverse reactions. The development of fully human mAbs has significantly reduced their immunogenic potential and it has improved their tolerability, compared to earlier chimeric or humanized mAbs. Nevertheless, even human mAbs maintain the potential for adverse reactions, such as anaphylactic reactions and infusion related reactions [Gklinos, P.; Papadopoulou, M.; Stanulovic, V.; Mitsikostas, D. D.; Papadopoulos, D. Monoclonal Antibodies as Neurological Therapeutics. Pharmaceuticals 2021, 14, 92. https://doi.org/10.3390/ph1402 0092]. There are also situations in which the Fc-mediated effect associated with the use of a monoclonal antibody is undesirable. For instance, unwanted activation of Fc receptor-expressing cells may lead to toxicity through cytokine release. Given all of this, the use of full mAbs as therapeutic agents is less desirable and the field has moved to develop alternative antibody forms.

Some antibody fragments preserve the ability of combining with antigen, which can replace complete mAbs [Bitencourt, A. L. B., Campos, R. M., Cline, E. N., Klein, W. L., and Sebollela, A. (2020). Antibody Fragments as Tools for Elucidating Structure-Toxicity Relationships and for Diagnostic/Therapeutic Targeting of Neurotoxic Amyloid Oligomers. IJMS 21, 8920. doi:10.3390/ijms21238920.]. The use of antibody fragments has advantages since the size of antibody fragments is small, they can be very useful in imaging, and the manufacturing cost is not high.

### Syncleinopathies

Alpha-synuclein ( [symbol] Syn) is a protein found primarily inside neurons and other brain cells, and has also been found outside of cells when being passed from cell to cell. Although the normal function of [symbol] Syn remains controversial, misfolding, aggregation, abnormal accumulation, and secretion of [symbol] Syn are closely associated with neurologic disorders known as syncleinopathies also called synucleinopathic disorders [e.g., Parkinson's Disease (PD), dementia with Lewy bodies, and multiple system atrophy]. Antibodies against [symbol] Syn have been considered as candidates as therapeutics in PD and have advanced into human clinical trials. Prior to initiating clinical trials, the efficacy of one such antibody, prasinezumab, was evaluated in various cellular and animal models of [symbol] Syn-related disease. In [symbol] Syn transgenic mice, the murine version of prasinezumab reduced the appearance of alpha synuclein pathology, protected synapses and halted the worsening of behavioral phenotypes. However, current approaches rely on antibody-based therapeutics reaching the brain, entering into neuronal cells, and affecting the pathology of PD by binding to its cognate antigen that is predominantly an intracellular protein. This pathway has proven challenging if not impossible for many antibody-based therapeutics.

### Alzheimer's Disease

Alzheimer's disease (AD) is characterized by progressive memory deficits and cognitive impairments. There is no cure for AD, and so far, treatments only slow the progression of the disease and ameliorate some of the symptoms. The 4 kDa Aβ peptide resulting from the cleavage of the amyloid precursor protein is considered a potential target for AD therapy [Karran, E.; Mercken, M.; De Strooper, B. The amyloid cascade hypothesis for Alzheimer's disease: An appraisal for the development of therapeutics. Nat. Rev. Drug Discov. 2011, 10, 698-712]. This peptide is prone to self-aggregation, forming neurotoxic species, and therapies are aimed at reversing the formation of these aggregates [Grill, J. D.; Cummings, J. L. Current therapeutic targets for the treatment of Alzheimer's disease. Expert Rev. Neurother. 2010, 10, 711-728]. Although clinical trials involving active immunization therapies using Aβ peptide were able to clear away toxic plaques and slowed cognitive decline among treated patients compared to control groups, they had undesirable side effects induced by T-cell-mediated and/or Fc-mediated meningoencephalitis and were abandoned [Check, E. Nerve inflammation halts trial for Alzheimer's drug. Nature 2002, 415, 462.; Orgogozo, J. M.; Gilman, S.; Dartigues, J. F.; Laurent, B.; Puel, M.; Kirby, L. C.; Jouanny, P.; Dubois, B.; Eisner, L.; Flitman, S.; et al. Subacute meningoencephalitis in a subset of patients with AD after abeta42 immunization. Neurology 2003, 61, 46-54]. Many second generation anti-amyloid mAbs have also been studied and undergone clinical trials. However, many of these clinical trials were terminated due to lack of positive results [Tian Hui Kwan, A., Arfaie, S., Therriault, J., Rosa-Neto, P., and Gauthier, S. (2020). Lessons Learnt from the Second Generation of Anti-amyloid Monoclonal Antibodies Clinical Trials. Dement Geriatr. Cogn. Disord. 49, 334-348. doi:10.1159/000511506].

Alternative strategies that involve engineered antibodies that do not contain the Fc fragment are attractive alternatives. Thus, scFvs have evolved as potential therapeutic venues for the treatment of AD [Robert, R.; Wark, K. L. Engineered antibody approaches for Alzheimer's disease immunotherapy. Arch. Biochem. Biophys. 2012, 526, 132-138].

The most prominent mode of delivery is through the use of adeno associated virus (AAV) as the delivery vehicle. Successful delivery using rAAVs expressing recombinant Aβ antibodies has been demonstrated in mice with direct intracranial injection. However, immunotherapy through recombinant AAV (rAAV) can cause hemorrhage caused by injection into ventricles [Kou, J., Kim, H., Pattanayak, A., Song, M., Lim, J.-E., Taguchi, H., et al. (2011). Anti-Amyloid-β Single-Chain Antibody Brain Delivery via AAV Reduces Amyloid Load but May Increase Cerebral Hemorrhages in an Alzheimer's Disease Mouse Model. Jad 27, 23-38. doi:10.3233/JAD-2011-110230]. Immunogenicity may also result when utilizing AAVs as delivery vehicles, thereby limiting multiple administrations that are needed for disorders of long duration.

### Huntington's Disease

Huntington's Disease (HD) is a neurodegenerative disease that is characterized by abnormal folding and proteolytic cleavage of the mutant huntington protein (mHTT) to N-terminal fragments, leading to formation of aggregates and neuronal and neuropil inclusion bodies in the brain. Because HD is a progressive genetic disorder with death occurring 10-20 years after diagnosis, early intervention therapies may significantly improve patient quality of life by slowing and/or reversing the course of the disease. To reduce the levels of mHTT, RNAi interference and protein-based techniques have been employed. RNAi-based approaches may have limitations when it comes to off-target toxic effects and non-specificity for the mutant protein [Butler, D. C.; McLear, J. A.; Messer, A. Engineered antibody therapies to counteract mutant huntingtin and related toxic intracellular proteins. Prog. Neurobiol. 2012, 97, 190-204]. ScFvs show less off-target effects and can target the mHTT protein based on conformational differences with the wild-type protein. Such scFvs have been generated that preferentially bind to different regions in the amino terminal of mHTT fragments compared to the full HTT and led to a reduction of aggregates. However, the neuroprotective effect weakened with the severity of disease at time of injection, and with age beyond 6 months, although it did not disappear entirely. [Butler D C, Messer A (2011) Bifunctional Anti-Huntingtin Proteasome-Directed Intrabodies Mediate Efficient Degradation of Mutant Huntingtin Exon 1 Protein Fragments. PLoS ONE 6(12): e29199]. Thus, additional optimization of scFv is required for this intrabody to be of future use in clinical applications for HD.

The normal mode of scFv delivery for HD therapy has been intracranial injections using AAV virus in mouse models [Butler, D. C.; Messer, A. Bifunctional anti-huntingtin proteasome-directed intrabodies mediate efficient degradation of mutant huntingtin exon 1 protein fragments. PLoS One 2011, 6, e29199.; Snyder-Keller, A.; McLear, J. A.; Hathorn, T.; Messer, A. Early or late-stage anti-N-terminal huntingtin intrabody gene therapy reduces pathological features in B6.HDR6/1 mice. J. Neuropathol. Exp. Neurol. 2010, 69, 1078-1085.]. Moreover, Southwell et al. tested AAV-mediated VL12.3 (also an anti-N-terminal intrabody) delivery in both R6/2 HTT Exon1 transgenic mice, and a model made by injecting mHTT Exon1 using a lentivirus. Lentivirus drove very high expression of mHTT Exon1 and co-administration of VL12.3 improved behavior and neuropathology [Southwell, A. L., et al., 2009. Intrabody gene therapy ameliorates motor, cognitive, and neuropathological symptoms in multiple mouse models of Huntington's disease. J. Neurosci. 29, 13589-13602]. However, this intrabody actually modestly accelerated disease in the R6/2 model, possibly due to higher levels of antigen-antibody complex in the nucleus of transduced cells [Southwell, A. L., et al., 2008. Intrabodies binding the proline-rich domains of mutant huntingtin increase its turnover and reduce neurotoxicity. J. Neurosci. 28, 9013-9020

### Amyotrophic Lateral Sclerosis

Amyotrophic lateral sclerosis (ALS) is a neurodegenerative disease characterized by the selective loss of motor neurons (motor neurons). While the majority of ALS cases appear to be sporadic, up to 20% of cases have been found to have genetic background and ˜20% of familial ALS cases are caused by mutations in Cu/Zn superoxide dismutase (SOD1). Mutant (mt) SOD1-induced disease results from a gain-of-function rather than loss-of-function as evidenced by the following: some FALS-inducing mtSOD1s have full dismutase activity; mice that carry a mtSOD1 transgene develop ALS despite the mouse's normal endogenous SOD1 activity; deletion of SOD1 does not cause an ALS-like disease in mice. While the toxicity of mtSOD1 is not fully understood, misfolding and aggregation of mtSOD1 is a consistent feature of the pathology of mtSOD1-induced ALS in patients and mtSOD1 transgenic rodent models, and has been proposed to underlie the basis for motor neuron degeneration. Aggregates of mtSOD1 could cause toxicity by: sequestering SOD1-binding proteins that are critical to the viability of motor neurons; exposing a toxic domain of SOD1; or increasing ER stress and thereby overwhelming the unfolded protein response.

There are a number of possible mechanisms by which mtSOD1 antibodies could prolong survival of mtSOD1 transgenic mice: interference with aggregation, thereby preventing the sequestration of proteins that are important for motor neuron survival; masking a toxic domain of mtSOD1 that is exposed when SOD1 is misfolded; change in the conformation of misfolded mtSOD1, thereby attenuating the mutant's toxicity and down-regulating expression of mtSOD1. Intraventricular infusion of anti-SOD1 monoclonal antibodies (mAbs) into familial ALS transgenic mice significantly prolonged survival Gros-Louis, F., et al., 2010. Intracerebroventricular infusion of monoclonal antibody or its derived Fab fragment against misfolded forms of SOD1 mutant delays mortality in a mouse model of ALS. J. Neurochem. 113, 1188-1199; Urushitani, M., et al., 2007. Therapeutic effects of immunization with mutant superoxide dismutase in mice models of amyotrophic lateral sclerosis. Proc. Natl. Acad. Sci. U.S.A. 104, 2495-2500]. These studies demonstrated that antibodies can be produced that are mtSOD1-specific and therapeutically effective in ALS. But here also the means of delivery was intracerebroventricular infusion, which is not practical for use in the clinical setting.

Use of Adeno Associated Viruses as Delivery Vehicles for scFvs as Therapeutic Agents

Despite the success in murine models using rAAVs expressing recombinant Aβ antibodies administered by intracranial injection, the delivery of AD therapeutics in humans is more likely to require a less invasive, administration route. Alternative delivery methods have been investigated. For example, AAV delivery of scFv by the Intra-CSF administration has also been used in mice.

Intravenous AAV deliveries have been tried in the past. Although some AAV serotypes have been reported to be efficient when transducing the CNS upon systemic delivery, concerns still remain regarding the ability of AAV to cross the BBB efficiently. Highest efficacy rates were obtained in newborn animals, whereas there is a limited BBB penetration in adult animals.

Regardless of BBB passage, main limitations inherent to systemic deliveries can be broadly summarized as (i) need for high volume of AAV to be injected, with high titration levels, (ii) undesired off-target effects, in particular potential liver toxicity, and (iii) limited CNS transduction, at least when relying on most of the currently available AAV capsid variants.

Thus, based upon the known limitations and immunogenicity concerns of using AAV as a delivery system, including for intrabodies such as scFv antibodies against diseases in the brain, AAV would not be the preferred method of delivery. Furthermore, the literature teaches away from intravenous administration of the AAV delivery system since intravenous administration of AAV-scFv, is known to result in immunogenicity and off-target effects in peripheral organs.

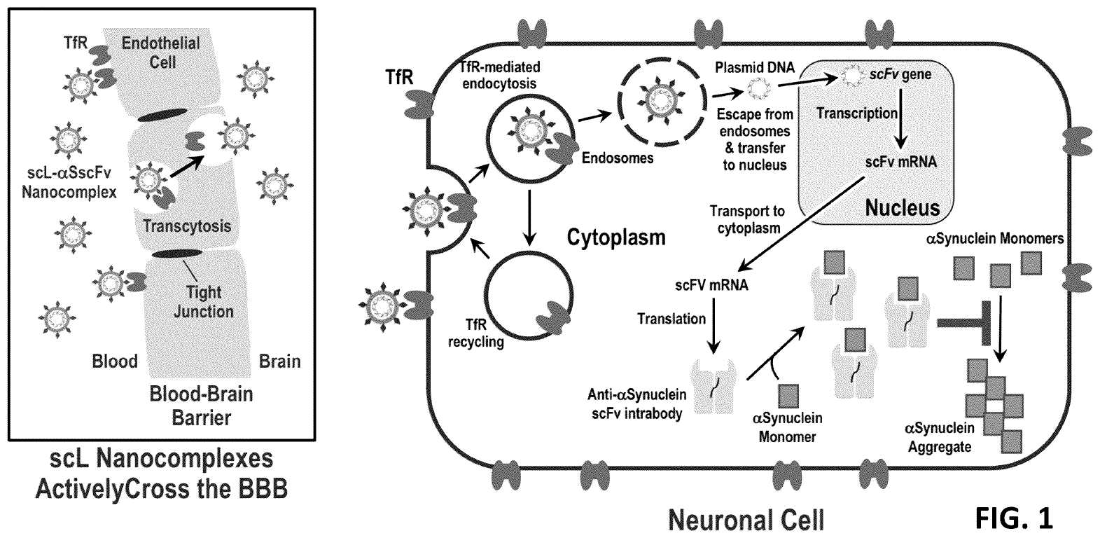

In contrast, the scL nanocomplex as described herein, efficiently crosses the BBB, and also efficiently delivers the payload into neuronal cells in the brain (see FIGS. 1, 3 and 4). This includes when the payload is an antibody or intrabody itself, as well as a plasmid DNA that encodes the subunits of the intrabody such that the intrabody will assemble and fold correctly in the cell resulting in biological activity against the target protein(s).

### Delivery of Intrabodies for Use in Treating Neurologic Disorders

Delivery of intrabody-based therapies, as either the protein itself or as plasmid DNA to form the intrabodies, requires systematic administration and delivery of the therapy across the blood-brain barrier and into neuronal cells. As described herein, in has been unexpectedly and surprisingly found that the nanocomplexes of this invention can not only cross the blood brain barrier but can also deliver the payload into neuronal cells in the brain, including the deep brain and thus provide efficacious effects against neurological disorders, including, but not limited to synucleinopathic disorders (including Parkinson's Disease), Alzheimer's Disease, Amyotrophic Lateral Sclerosis (ALS) disease and Huntington's disease.

In embodiments, provided herein is a method of treating a neurologic disorder in a patient, comprising: providing a transferrin receptor-targeted cationic liposomal complex, comprising: a cationic liposome; a transferrin receptor-targeting molecule that is complexed with the cationic liposome, but is not chemically conjugated to the cationic liposome, and wherein the targeting moiety does not comprise a lipid tag; and an antibody or a nucleic acid encoding an intrabody against a protein associated with the neurologic disorder; and administering the transferrin receptor-targeted cationic liposomal complex to the patient to treat the disorder.

As used herein, “neurologic disorder” includes the various neurologic disorders and diseases described herein that affect the brain as well as the nerves found throughout the human body and the spinal cord.

In embodiments, the methods provided herein are able to deliver a plasmid comprising a nucleic acid encoding an intrabody (e.g., an scFv molecule) targeting the specific protein related to a neurological disorder (including but not limited to, anti-Aβ scFv or anti-Aβ42 for Alzheimer's Disease, Anti-SOD1 scFv for Amyotrophic Lateral Sclerosis, anti-huntingtin scFv for Huntington's Disease or anti-α synuclein scFv for Parkinson's Disease) and can result in efficacious effects against neurological disorders, including, but not limited to synucleinopathic disorders (including Parkinson's Disease), Alzheimer's Disease, Amyotrophic Lateral Sclerosis (ALS) disease and Huntington's disease. In embodiments, the nucleic acid encodes a protein which is a nanobody targeted to a neuronal postsynaptic scaffolding protein, Homer1, that is present at partially overlapping sets of excitatory synapses, and is associated with Fragile X Syndrome (FXS) and Deafness Autosomal Dominant 68 (DFNA68).

In further embodiments, provided herein is a method of treating a neurologic disorder in a patient, comprising: providing a transferrin receptor-targeted cationic liposomal complex, comprising: a cationic liposome; a transferrin receptor-targeting molecule that is complexed with the cationic liposome, but is not chemically conjugated to the cationic liposome, and wherein the targeting moiety does not comprise a lipid tag; and an intrabody against a protein associated with the neurologic disorder; and administering the transferrin receptor-targeted cationic liposomal complex to the patient to treat the disorder.

In exemplary embodiments, the intrabody is a single chain FV antibody fragment (scFv), an antibody (IgG), an Fab, and F(ab′)2, a mono- or bi-specific Fab2, a Tri-specific Fab3, a monovalent IgG, a bi- or tri-specific diabody, a minibody, an IgNAR a V-NAR, a hclIgG or a VhH fragment.

Although monoclonal antibodies are being widely used as therapeutics, these antibodies largely recognize antigens that are either completely outside of cells or that reside on the exterior surface of the plasma membrane. Antibody-based therapeutics do not readily enter cells to reach intracellular target antigens. The challenge of using antibodies as therapeutics against targets inside cells of the brain is even more daunting, because access to the brain as a whole is impeded by the blood-brain barrier (BBB). The BBB is a security measure consisting of a tightly regulated gateway formed by endothelial cells lining brain blood vessels that allows only certain molecules to pass from the bloodstream to the brain. Some relatively large proteins (e.g., transferrin or insulin) are actively moved across the BBB by a process known as receptor-mediated transcytosis. However, most proteins, including antibodies, are denied brain entry by the BBB.

As used herein, the term “intrabody” or “intrabodies” refers to an antibody that binds a target intracellular protein and works within a cell (as opposed to binding a target protein on the outer surface of a cell). The advantages of using intrabodies for neurodegenerative diseases involving misfolding of proteins or aggregation of proteins are often related to the fact that, pathogenic misfolded proteins, often originate intracellularly and the aggregated proteins are seen in the cytoplasm of neuronal cells.

In the case of □Syn, the protein moves from one cell to another, and prasinezumab has its apparent therapeutic impact by interacting with extracellular □Syn. Nonetheless, it remains the case that □Syn originates and accumulates intracellularly. An intrabody against □Syn can both reduce production of □Syn in the cells of origin and “immunize” recipient cells against incoming □Syn originating in another cell.

In the case of Alzheimer's Disease, the Aβ protein self-aggregates, forming neurotoxic species. An intrabody against αB can prevent or reverse the formation of these aggregates.

With Huntington's Disease, it is abnormal folding and proteolytic cleavage of the mutant huntingtin protein (mHTT) to N-terminal fragments, which leads to formation of aggregates and neuronal and neuropil inclusion bodies in the brain. Intrabodies which can result in the reduction of such aggregates may significantly improve patient quality of life by slowing and/or reversing the course of the disease.

Amyotrophic Lateral Sclerosis is different in that it is associated with gain of function caused by mutations in Cu/Zn superoxide dismutase (SOD1). Intrabodies targeted to mtSOD1 can slow progression of the disease by interfering with mtSOD1 induced aggregation, thereby preventing the sequestration of proteins that are important for motor neuron survival; masking a toxic domain of mtSOD1 that is exposed when SOD1 is misfolded; change in the conformation of misfolded mtSOD1, thereby attenuating the mutant's toxicity and down-regulating expression of mtSOD1.

Nucleic acids encoding intrabodies for use in the methods described herein include nucleic acids that encode full antibodies, antibody fragments (Fab fragments), single domain antibodies (such as the camelid family), single chain antibodies and single chain antibody fragments (single chain Fv (scF) fragments), so long as their target for treatment of a disorder is an intracellular protein.

The three main kinds of antibody fragments described herein (single-chain Fv (scFv) fragments, Fab fragments and single domain antibody fragments) are all devoid of the Fc portion of the antibody that is more responsible for adverse reactions to antibody-based therapeutics. A single-chain Fv fragment mimics the combination of the variable heavy (VH) and variable light (VL) of an IgG by linking these domains via a flexible hinge sequence. Another advantage to the use of scFv fragments is that they can be expressed intracellularly from a single gene from which the secretory signal of the parental IgG has been removed. The general size of scFv (˜250 amino acids) is only about 15% that of an IgG.

As described herein, the transferrin receptor-targeted cationic liposome complex is capable of crossing the BBB via the normal physiological process of transcytosis mediated by the transferrin receptor (TfR) [also called scL nanocomplexes (for single chain Liposomes) or Tf-Lip (for transferrin Liposomes)]. Once in the brain, the TfR-targeted nanocomplexes are taken up by neuronal cells via TfR-mediated endocytosis. When the payload is a nucleic acid molecule encoding a gene (including a plasmid), that gene is able to avoid the endothelial cells of the BBB, escape from the endosomes of the neuronal cells, and enter the cytoplasm and ultimately the nucleus, allowing for expression, and the gene-encoded protein is made in situ in the cytoplasm over a period of days. Delivery of a gene with a strong promoter allows for production of a significantly higher amount of protein than could be achieved via i.v. injection of a protein therapeutic, especially if the protein does not efficiently cross the BBB as in the case of antibodies.

FIG. 1 summarizes the approach described herein using as an example αSyn. The approach of this invention would be essentially the same for the use of any intrabody against any neurologic disease including but not limited to Alzheimer's Disease, Amyotrophic Lateral Sclerosis (ALS) disease and Huntington's disease. The methods include delivery into neuronal cells of nucleic acids encoding intrabodies, such as single domain antibodies and single chain Fv antibody fragments against proteins associated with such neurologic disorders, suitably using a liposomal complex that crosses the blood brain barrier and can deliver the payload into CNS cells. In this example, what is illustrated is anti- [symbol] Syn intrabodies' ability to block the aggregation of [symbol] Syn for therapeutic effect in Parkinson's disease and other synucleinopathies. Various mechanisms by which [symbol] Syn intrabodies have a therapeutic effect are envisioned. The [symbol] Syn intrabody can bind to monomers of [symbol] Syn in the cytoplasm and affect its aggregation in the cytoplasm as depicted in FIG. 1. In addition, these intrabodies can also impede release of [symbol] Syn from neuronal cells, “immunize” recipient cells against incoming [symbol] Syn, and/or mediate degradation of [symbol] Syn monomers or aggregates. The ability of intracellular antibody fragments may be based on multiple mechanisms of action.

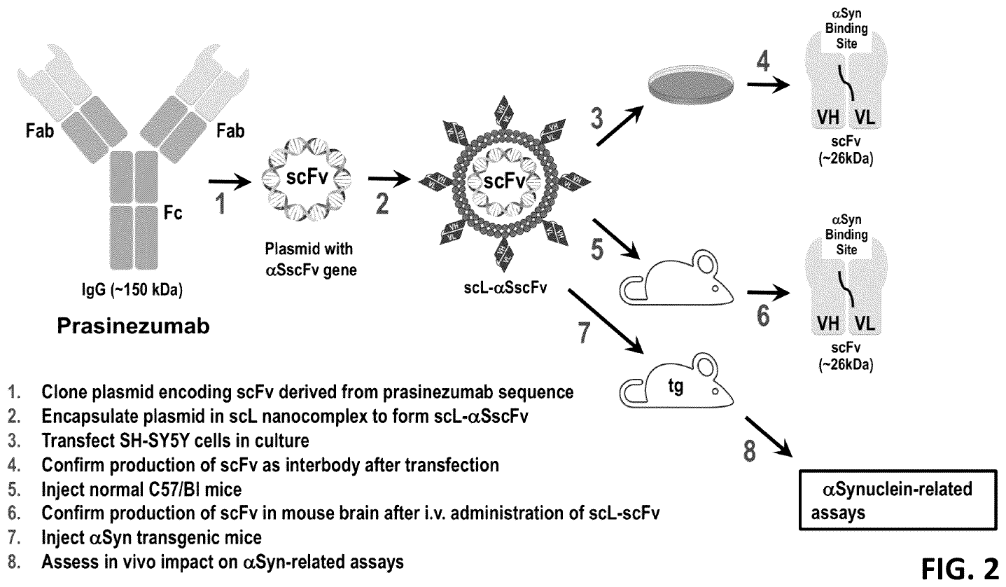

As described in FIG. 2 and herein, αSyn is used as an example. However, the approach is the same irrespective of the specific intrabody to be produced or the neurologic disease targeted. A gene encoding an intrabody, such as an scFv against [symbol] Syn that is derived from the sequence of prasinezumab, can be delivered using the methods described herein. An scL nanocomplex (termed scL- [symbol] scFv) carrying a plasmid with the gene encoding the desired intrabody can be delivered intravenously. Methods are described herein for the production of the intrabody gene, and transfection of neuronal cells with scL-αSscFv confirming intracellular production of the intrabody. Various strains of [symbol] Syn transgenic (tg) mice exist, and assays to assess the impact of the therapeutics are available. These include immunohistochemistry for [symbol] Syn in cortex and hippocampus, assessment of neuroinflammation, extraction and quantification of [symbol] Syn protein, and assessment of behaviors reflective of neurological impairment in the [symbol] Syn tg mice.

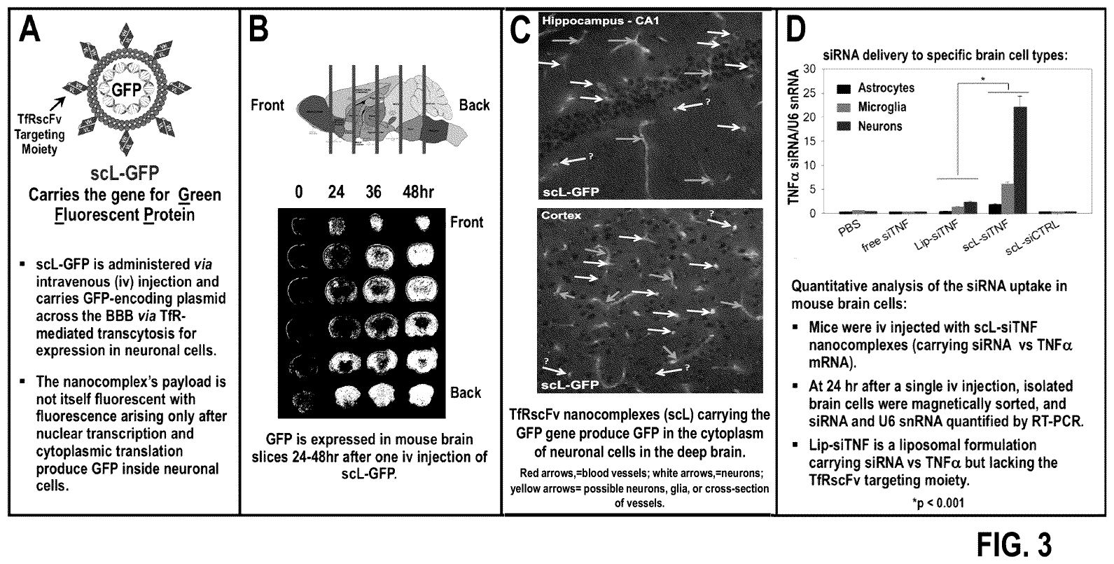

FIG. 3 demonstrates the ability of systemically (intravenous) administered scL nanocomplexes to cross the BBB and deliver exogenous genes for cytoplasmic expression in neuronal cells. Panel A is a schematic of the scL nanocomplex carrying the gene for green fluorescent protein (GFP). Panel B shows that this nanocomplex crosses the BBB, and enters cells with the encoded GFP protein only produced inside the cell. GFP has been shown to be expressed throughout the brain. Panel C demonstrates that a GFP gene delivered via TfRscFv-mediated transcytosis plus endocytosis leads to cytoplasmic expression of the gene product (as one would predict if the gene were encoding an anti-αSyn intrabody). Panel D uses an scL-delivered siRNA to show that the payload is delivered to neurons, astrocytes and microglia following systemic (i.v.) administration.

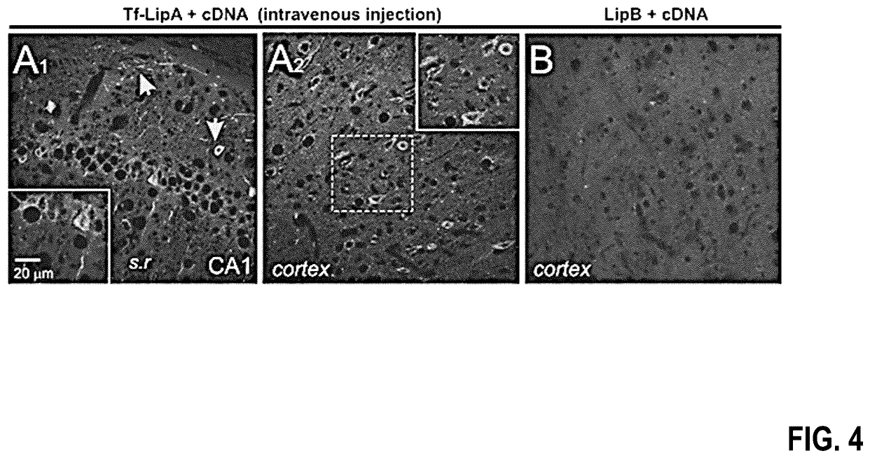

FIG. 4 demonstrates that Transferrin itself (Tf) can be used as the targeting moiety for the nanocomplexes. Panel A demonstrates that after systemic (intravenous) administration of the Tf-targeted nanocomplex carrying the gene for green fluorescent protein (GFP) crosses the BBB, enters neurons in the deep brain and that the GFP protein is expressed in the cytoplasm. In contrast, there is no neuronal targeting when the nanocomplexes without the Tf ligand is injected (Panel B).

In exemplary embodiments the exogenous genes encode intrabodies targeting genes associated with, but not limited to, synucleinopathic disorders (including Parkinson's Disease), Alzheimer's Disease, Amyotrophic Lateral Sclerosis (ALS) disease and Huntington's disease. Also, in another embodiment the gene encodes a protein which is a nanobody targeted to a neuronal postsynaptic scaffolding protein (Homer1) that is present at partially overlapping sets of excitatory synapses, and is associated with Fragile X Syndrome (FXS) and Deafness Autosomal Dominant 68 (DFNA68).

In embodiments, the method of preparing the Transferrin Receptor (TfR) targeted cationic liposomal complex, i.e., the delivery systems described herein, suitably comprise preparing a lipid solution comprising one or more cationic lipids in ethanol, injecting the mixture of lipids into an aqueous solution, thereby forming a cationic liposome, mixing the cationic liposome with a targeting moiety to form the targeted cationic liposome, wherein the targeting moiety is directly complexed with, but not chemically conjugated to, the cationic liposome. The targeting moiety can be transferrin, the natural ligand of the TfR or an antibody or antibody fragment recognizing the TfR. The antibody or antibody fragment is mixed with the cationic liposome at room temperature and at a protein:lipid ratio in the range of about 1:20 to about 1:40 (w:w).

Methods for preparing the targeting moiety-targeted cationic liposome complexes are described, for example, in U.S. 2005/0002998, the disclosure of which is incorporated by reference herein in its entirety.

It has been unexpectedly found that no extrusion or sonication is required to form the liposomes and the targeted cationic liposomes having the desired size and Zeta Potential characteristics, according to the methods described herein. In embodiments, evaporation, sonication, milling and/or extrusion of the liposomes is specifically excluded from the disclosed methods. In further embodiments, the methods of preparing targeted cationic liposomes described throughout suitably consist of or consist essentially of the recited elements. In such embodiments, addition of steps such as evaporation, sonication and/or extrusion, are considered a material alteration to such methods and thus are specifically excluded from such methods that consist essentially of the recited elements.

In embodiments, the nucleic acid to be delivered is one or more genes, or polynucleotides, such as plasmid DNA, DNA fragment, oligonucleotide, oligodeoxynucleotide, antisense oligonucleotide, chimeric RNA/DNA oligonucleotide, RNA, siRNA, mRNA, mRNA, ribozyme, viral particle, growth factor, cytokine, antibody, antibody fragment, immunomodulating agent, or other protein, including proteins. Exemplary therapeutic agents are nucleic acid molecules, preferably DNA, siRNA, mRNA, or miRNA molecules. A suitable DNA molecule is a plasmid which encodes an intrabody, such as a single domain antibody, or a single chain FV antibody fragment (scFV), targeted to a protein associated with a neurological disorder, including, but not limited to, synucleinopathic disorders (including Parkinson's Disease), Alzheimer's Disease, Amyotrophic Lateral Sclerosis (ALS) disease and Huntington's disease. In another embodiment the plasmid(s) comprise nucleic acid(s) that encodes an intrabody which is a complete antibody (IgG), an Fab, and F(ab′)₂, a mono- or bi-specific Fab₂, Tri-specific Fab₃, A monovalent IgG, a bi- or tri-specific diabody, ann scFv-Fc fragment, a minibody, an IgNAR a V-NAR, a hclIgG or a VhH fragment. In another embodiment the nucleic acid encodes a protein which is a nanobody targeted to neuronal postsynaptic scaffolding protein Homer1, that is present at partially overlapping sets of excitatory synapses, and is associated with Fragile X Syndrome (FXS) and Deafness Autosomal Dominant 68 (DFNA68).

Exemplary nucleic acids in plasmids that can be delivered using the cationic liposomal complexes include the following given below. However, it should be understood that any nucleic acid sequence encoding a suitable intrabody can be used in the methods and complexes described herein.

1) Anti-Alpha Synuclein scFv Antibody
DNA: Synthetic construct anti-alpha synuclein scFv antibody gene, complete cds. GenBank: EU079027.1 (798 bp) ncbi.nlm.nih.gov/nuccore/EU079027.1
Protein: anti-alpha synuclein scFv antibody [synthetic construct] GenBank: ABW74530.1 (265 aa) ncbi.nlm.nih.gov/protein/abw74530.1
Reference: Lynch, S. M., Zhou, C. and Messer, A. An scFv intrabody against the nonamyloid component of alpha-synuclein reduces intracellular aggregation and toxicity, J. Mol. Biol. 377 (1), 136-147 (2008) pubmed.ncbi.nlm.nih.gov/18237741/

EU079027.1 Synthetic construct anti-alpha synuclein scFv antibody gene, complete cds (798 bp)

(SEQ ID NO: 1)
ATGTCTAGCCAGGTACAGCTGCAGCAGTCAGGTCCAGGACTGGTG
AAGCCCTCGCAGACCCTCTCACTCACCTGTGCCATCTCCGGGGA
CAGTGTCTCTAGCAACAGTGCTGCTTGGAACTGGATCAGGCAGTC
CCCATCGAGAGGCCTTGAGTGGCTGGGAAGGACATTCTACAGGTC
CAAGTGGTATAATGATTATGCAGCATCTGTGAAAAGTCGAATAAC
CATCGACCCAGACACATCCAAGAACCAGTTCTCCCTGCAGCTGAA
CTCTGTGACTCCCGAAGACACGGCTGTATATTACTGTACACGACA
AAACTTGGCTGGTCCCTTTGACTCCTGGGGCCAGGGTACCCTGGT
CACCGTCTCCTCAGGAATTCTAGGATCCGGTGGCGGTGGCAGCGG
CGGTGGTGGTTCCGGAGGCGGCGGTTCTGAAATTGTGATGACACA
GTCTCCAGGCACCCTGTCTTTGTCTCCAGGGGAAAGAGCCACCCT
CTCCTGTAGGGCCAGTCAGAGTGTTAGCAGCAACTACTTAGCCTG
GTACCAGCAGAAAGCTGGCCAGGCTCCCAGGCTCCTCATCAGTGG
TGCATCCAGCAGGGCCACTGGCATCCCAGACAGGTTCAGTGGCAG
TGGGTCTGGGACAGACTTCACTCTCACCATCAGCAGACTGGAGCC
TGAAGATTTTGCAGTGTATTACTGTCAGCAGTATGGTAGCTCAAC
AGCTTTCGGCCCTGGGACCAAAGTGGATATCAAATCCGGAATTCT
AGAACAAAAGCTTATTTCTGAAGAAGACTTGTAA

2) Anti-Alpha Synuclein scFv Antibody
DNA: Synthetic construct anti-oligomeric synuclein single-chain Fv antibody D5E gene, complete cds GenBank: JX442980.1 (729 bp) ncbi.nlm.nih.gov/nuccore/EU079027.1
Protein: anti-oligomeric synuclein single-chain Fv antibody D5E [synthetic construct] GenBank: AFR23376.1 (242 aa) ncbi.nlm.nih.gov/protein/afr23376.1
Reference: Emadi S, Barkhordarian H, Wang M S, Schulz P, Sierks M R. Isolation of a human single chain antibody fragment against oligomeric alpha-synuclein that inhibits aggregation and prevents alpha-synuclein-induced toxicity. J Mol Biol. 2007; 368(4):1132-1144. pubmed.ncbi.nlm.nih.gov/17391701/.

JX442980.1 Synthetic construct anti-oligomeric synuclein single-chain Fv antibody D5E gene, complete cds (729 bp)

(SEQ ID NO: 2)
ATGGCCGAGGTGCAGCTGTTGGAGTCTGGGGGAGGCTTGGTACAG
CCTGGGGGGTCCCTGAGACTCTCCTGTGCAGCCTCTGGATTCAC
CTTTAGCAGCTATGCCATGAGCTGGGTCCGCCAGGCTCCAGGGAA
GGGGCTGGAGTGGGTCTCATCGATTGGTCAGAAGGGTGGTGGTAC
AGAGTACGCAGACTCCGTGAAGGGCCGGTTCACCATCTCCAGAGA
CAATTCCAAGAACACGCTGTATCTGCAAATGAACAGCCTGAGAGC
CGAGGACACGG
CCGTATATTACTGTGCGAAACATTTTGAGAATTTTGACTACTGGG
GCCAGGGAACCCTGGTCACCGTCTCGAGCGGTGGAGGCGGTTCA
GGCGGAGGTGGCAGCGGCGGTGGCGGGTCGACGGACATCCAGATG
ACCCAGTCTCCATCCTCCCTGTCTGCATCTGTAGGAGACAGAGTC
ACCATCACTTGCCGGGCAAGTCAGAGCATTAGCAGCTATTTAAAT
TGGTATCAGCAGAAACCAGGGAAAGCCCCTAAGCTCCTGATCTAT
GCTGCATCCCATTTGCAAAGTGGGGTCCCATCAAGGTTCAGTGGC
AGTGGATCTGGGACAGATTTCACTCTCACCATCAGCAGTCTGCAA
CCTGAAGATTTTGCAACTTACTACTGTCAACAGACGCGTAGGCCG
CCTTCTACGTTCGGCCAAGGGACCAAGGTGGAAATCAAACGGTAA

3) Anti-Alpha Synuclein scFv Antibody
DNA: Synthetic construct anti-synuclein single-chain Fv antibody 10H gene, complete cds GenBank: JX430806.1 (768 bp) ncbi.nlm.nih.gov/nuccore/JX430806.1
Protein: anti-synuclein single-chain Fv antibody 10H [synthetic construct] GenBank: AFR23374.1 (255 aa) ncbi.nlm.nih.gov/protein/afr23374.1
Reference: Emadi S, Kasturirangan S, Wang M S, Schulz P, Sierks M R. Detecting morphologically distinct oligomeric forms of alpha-synuclein. J Biol Chem. 2009; 284(17):11048-11058. pubmed.ncbi.nlm.nih.gov/19141614/

JX430806.1 Synthetic construct anti-synuclein single-chain Fv antibody 10H gene, complete cds (768 bp)

(SEQ ID NO: 3)
ATGGCCGAGGTGCAGCTGTTGGAGTCTGGGGGAGGCTTGGTACAG
CCTGGGGGGTCCCTGAGACTCTCCTGTGCAGCCTCTGGATTCAC
CTTTAGCAGCTATGCCATGAGCTGGGTCCGCCAGGCTCCAGGGAA
GGGGCTGGAGTGGGTCTCAAATATTAGTAGTGCAGGGAAGGGGCT
GGAGTGGGTCTCAAGTATTGATGATTCTGGTGCTTCTACATATTA
CGCAGACTCCGTGAAGGGCCGGTTCACCATCTCCAGAGACAATTC
CAAGAACACGCTGTATCTGCAAATGAACAGCCTGAGAGCCGAGGA
CACGGCCGTATATTACTGTGCGAAAGATTCTGCTTCTTTTGACTA
CTGGGGCCAGGGAACCCTGGTCACCGTCTCGAGCGGTGGAGGCGG
TTCAGGCGGAGGTGGCAGCGGCGGTGGCGGGTCGACGGACATCCA
GATGACCCAGTCTCCATCCTCCCTGTCTGCATCTGTAGGAGACAG
AGTCACCATCACTTGCCGGGCAAGTCAGAGCATTAGCAGCTATTT
AAATTGGTATCAGCAGAAACCAGGGAAAGCCCCTAAGCTCCTGAT
CTATACTGCATCCAGTTTGCAAAGTGGGGTCCCATCAAGGTTCAG
TGGCAGTGGATCTGGGACAGATTTCACTCTCACCATCAGCAGTCT
GCAACCTGAAGATTTTGCAACTTACTACTGTCAACAGTCTGCTGC
TAGTCCTTCTACGTTCGGCCAAGGGACCAAGGTGGAAATCAAACG
GTAA

### Anti-Beta Amyloid (for Alzheimer's Disease)

2) Anti-Abeta42 scFv Antibody
DNA: Synthetic construct clone ht7 anti-Abeta42 single chain variable fragment antibody gene, complete cds GenBank: MG385840.1 (750 bp) ncbi.nlm.nih.gov/nuccore/MG385840.1
Protein: anti-Abeta42 single chain variable fragment antibody [synthetic construct] GenBank: AYC35246.1 (249 aa) ncbi.nlm.nih.gov/protein/ayc35246.1
Reference: Zhang Y, Huai Y, Zhang X, Song C, Cai J, Zhang Y. The Mode of Action of an Anti-Oligomeric Amyloid β-Protein Antibody Affects its Protective Efficacy. Neurotox Res. 2019; 35(2):304-317. pubmed.ncbi.nlm.nih.gov/30229545/

MG385840.1 Synthetic construct clone ht7 anti-Abeta42 single chain variable fragment antibody gene, complete cds (750 bp)

(SEQ ID NO: 4)
ATGCAGGTGCAGCTGGTGCAGTCTGGAGCTGAGGTGAAGAAGCCT
GGGGCCTCAGTGAAGGTCTCCTGCAAGGCTTCTGGTTACACCGT
TACCAGTTATGGTATCAGCTGGGTGCGACAGGCCCCTGGACAAGG
GCTTGAGTGGATGGGAGGGATCAACACCAATACTGGGAACCCAGC
GTATGCCCCGGGCTTCACAGGACGATTTGTCTTCTCCTTGGACAC
CTCTGTCAGCACGGCACACCTGCAGATCGGCAGCCTAAAGGCTGA
GGACACCGCCGTTTATTTCTGTGCGAGGAGTTCGAGTGGTTATCA
GGGTTCCTCCTACTACTTCGATATGGACGTCTGGGACCAAGGGAC
AACGGTCACCGTCTCTTCAGGTGGAGGCGGTTCTGGCGGAGGTGG
CTCAGGTGGTGGAGGATCGGATATCCAGATGACTCAGTCTCCATC
CTCCCTGTCTGCGTCTGTAGGAGACAGAGTCACCATCACTTGCCG
GGCAAGTCGGACCATTAGCAGTTATTTAAATTGGTATCAGCAGAA
ACCAGGGAAACCCCCTAAACTCCTGATCTATGCTGCATCCATTTT
ACAAAGTGGGGTCCCATCAAGGTTCAGTGGCAGTGTATCTGGGAC
ACATTTCACTCTCACCATCAGCAGTCTGCAACCTGAAGATTTTGC
AACCCACTACTGTCAACAGAGTTCCAGTACCCCGCTTACTTTCGG
CGGAGGGACCAAAGTGGAGATCAAACGTTGA

2) Anti-Abeta42 scFv Antibody
DNA: Synthetic construct anti-Abeta42 single chain variable fragment antibody gene, complete cds (711 bp) ncbi.nlm.nih.gov/nuccore/KM283423.1
Protein: anti-Abeta42 single chain variable fragment antibody [synthetic construct] GenBank: AIX97363.1 (236 aa) ncbi.nlm.nih.gov/protein/aix97363.1
Reference: Zhang Y, Chen X, Liu J, Zhang Y. The protective effects and underlying mechanism of an anti-oligomeric A042 single-chain variable fragment antibody. Neuropharmacology. 2015; 99:387-395. pubmed.ncbi.nlm.nih.gov/26256421/In vitro validation. No in vivo work.

KM283423.1 Synthetic construct anti-Abeta42 single chain variable fragment antibody gene, complete cds (711 bp)

(SEQ ID NO: 5)
ATGCAGGTGCAGCTGGTGCAGTCTGGAGCTGAGGTGAAGAAGCCT
GGGGCCTCAGTGAAGGTCTCCTGCAAGGCTTCTGGTTACACCGT
TACCAGTTATGGTATCAGCTGGGTGCGACAGGCCCCTGGACAAGG
GCTTGAGTGGATGGGAGGGATCAACACCAATACTGGGAACCCAGC
GTATGCCCCGGGCTTCACAGGACGATTTGTCTTCTCCTTGGACAC
CTCTGTCAGCACGGCACACCTGCAGATCGGCAGCCTAAAGGCTGA
GGACACCGCCGTTTATTTCTGTGCGAGGAGTTCGAGTGGTTATCA
GGGTTCCTCCTACTACTTCGATATGGACGTCTGGGGCCAAGGGAC
AACGGTCACCGTCTCTTCAGGTGGAGGCGGTTCTGGCGGAGGTGG
CTCAGGCGGTGGAGGATCGGATATCCAGATGACTCAGTCTCCATC
CTCCCTGTCTGCATCTGTAGGAGACAGAGTCATCATCACTTGCCG
GGCGAGTCAGGGCATTAGCAAGTATTTAGCCTGGTATCAGCAGAA
ACCAGGGAAAGCCCCTAAGGTCCTGATCGATGCTGCATCCAGTTT
GCAAAGTGGGGTCCCGTCAAGGCTCAGTGGCAGTGGATCTGGGAC
AGATTTCACTCTTACCATCAGCAGCCTGCAGCCGGAAGATTTTGC
AACTTATTACTGTCTACAAGATGACAGTTACCCTTGA

3) Anti-Abeta42 scFv Antibody
DNA: Synthetic construct anti-Abeta42 single chain variable fragment antibody (scFv) gene, complete cds GenBank: KJ866152.1 (720 bp) ncbi.nlm.nih.gov/nuccore/KJ866152.1
Protein: anti-Abeta42 single chain variable fragment antibody [synthetic construct]

GenBank: AII79400.1 (239 aa) ncbi.nlm.nih.gov/protein/aii79400.1
Reference: Zhang Y, Sun Y, Huai Y, Zhang Y J. Functional Characteristics and Molecular Mechanism of a New scFv Antibody Against A042 Oligomers and Immature Protofibrils [published correction appears in Mol Neurobiol. 2015 December; 52(3):1282-3]. Mol Neurobiol. 2015; 52(3):1269-1281. doi:10.1007/s12035-014-8910-7. a pubmed.ncbi.nlm.nih.gov/25330935/

KJ866152.1 Synthetic construct anti-Abeta42 single chain variable fragment antibody (scFv) gene, complete cds (720 bp)

(SEQ ID NO: 6)
ATGCAGGTGCAGCTGGTGGAGTCTGGGGGAGGCGTGGTCCAGCCT
GGGAAGTCCCTGAGACTCTCCTGTGCAGCCTCTGGATTCACCTT
CAGTAGTCAAGGCACGCACTGGGTCCGCCAGGCTCCAGGCAAGGG
GCTGGAGTGGGTGGCAGTTATACGGCGTGATGGAAGTAATATACA
CTATGCAGACTCCGTGAGGGGCCGATTCACCGTCTCCAGAGACAA
TTCCAAGAACACTCTGTATCTGGAAATCAACAGCCTGAGAGTTGA
GGACACGGCTGTCTATTTCTGCGCCAAGGGGGGGTCTTTTGATCT
CTGGGGCCAAGGGACAACGGTCACCGTCTCTTCAGGTGGAGGCGG
TTCTGGCGGAGGTGGCTCAGGCGGTGGAGGATCGGATATCCAGAT
GACTCAGTCTCCTTCATCCCTGTCAGCATCTGTAGGAGAGAGAGT
CACCATCACTTGCCGGGCGAGTCAGGCCATTATTAATTCTTTAGC
CTGGTATCAACAGAAACCAGGGAAAGCCCCTAAGCTCCTGATCTA
TGCTGCATCCAGTTTGCAAAGTGGGGTCCCATCAAGGTTCAGTGG
CAGTGGATCTGGGACAGATTTCACTCTCACCATCAGCAGTCTGCA
ACCTGAAGATTTTGCAACTTACTACTGTCAACAGAGTTACAGTAC
CCTGTGGACGTTCGGCCAAGGGACCAAAGTGGAGATCAAACGTTG
A

4) Anti-Abeta scFv Antibody
DNA: Synthetic construct immunoglobulin single-chain variable region scFv59 gene, complete cds GenBank: KF534723.1 (852 bp) ncbi.nlm.nih.gov/nuccore/KF534723.1
Protein: immunoglobulin single-chain variable region scFv59 [synthetic construct] GenBank: AHA61677.1 (283 aa) ncbi.nlm.nih.gov/protein/aha61677.1
Reference: Fukuchi K, Accavitti-Loper M A, Kim H D, et al. Amelioration of amyloid load by anti-Abeta single-chain antibody in Alzheimer mouse model. Biochem Biophys Res Commun. 2006; 344(1):79-86. pubmed.ncbi.nlm.nih.gov/16630540/

KF534723.1 Synthetic construct immunoglobulin single-chain variable region scFv59 gene, complete cds (852 bp)

(SEQ ID NO: 6)
ATGGAGTTTGGGCTGAGCTGGCTTTTTCTTGTGGCTATTTTAAAA
GGTGGTGTACAGTGTGAGGTGCAGCTGTTGGAGTCTGGGGGAGG
CTTGGTACAGCCTGGGGGGTCCCTGAGACTCTCCTGTGCAGCCTC
TGGATTCACCTTTAGCAGCTATGCCATGAGCTGGGTCCGCCAGGC
TCCAGGGAAGGGGCTGGAGTGGGTCTCAGCTATTAGTGGTAGTGG
TGGTAGCACATACTACGCAGACTCCGTGAAGGGCCGGTTCACCAT
CTCCAGAGACAATTCCAAGAACACGCTGTATCTGCAAATGAACAG
CCTGAGAGCCGAGGACACGGCCGTATATTACTGTGCGAAAAAGGG
TACGACGGATTTTGACTACTGGGGCCAGGGAACCCTGGTCACCGT
GTCGAGAGGTGGAGGCGGTTCAGGCGGAGGTGGCTCTGGCGGTGG
CGGATCTTCTGAGCTGACTCAGGACCCTGCTGTGTCTGTGGCCTT
GGGACAGACAGTCAGGATCACATGCCAAGGAGACAGCCTCAGAAG
CTATTATGCAAGCTGGTACCAGCAGAAGCCAGGACAGGCCCCTGT
ACTTGTCATCTATGGTAAAAACAACCGGCCCTCAGGGATCCCAGA
CCGATTCTCTGGCTCCAGCTCAGGAAACACAGCTTCCTTGACCAT
CACTGGGGCTCAGGCGGAAGATGAGGCTGACTATTACTGTAACTC
CTCTCTGACGCGTAGGCCCCATGTGGTATTCGGCGGAGGGACCAA
GCTGACCGTCCTAGGCGCGGCCGCAGATGACGATTCCGACGATGA
CTACAAGGACGACGACGACAAGCACCATCACCATCACCATTAG

### Anti-Copper Zinc Superoxide Dismutase 1 (Sod1) (for Als)

3) Anti-SOD1 scFv Antibody
DNA: Synthetic construct anti-copper zinc superoxide dismutase 1 intrabody single chain Fv antibody B12 gene, complete cds GenBank: KC845560.1 (765 bp) ncbi.nlm.nih.gov/nuccore/KC845560.1
Protein: anti-copper zinc superoxide dismutase 1 intrabody single chain Fv antibody B12 [synthetic construct] GenBank: AGK37120.1 (254 aa) ncbi.nlm.nih.gov/protein/agk37120.1
Reference: Ghadge G D, Pavlovic J D, Koduvayur S P, Kay B K, Roos R P. Single chain variable fragment antibodies block aggregation and toxicity induced by familial ALS-linked mutant forms of SOD1 [published correction appears in Neurobiol Dis. 2013 October; 58:115]. Neurobiol Dis. 2013; 56:74-78. pubmed.ncbi.nlm.nih.gov/23607939/

KC845560.1 Synthetic construct anti-copper zinc superoxide dismutase 1 intrabody single chain Fv antibody B12 gene, complete cds (765 bp)

(SEQ ID NO: 7)
ATGGACTACAAGGACGACGATGACAAGCTTCTGCCTGTGCTGACT
CAGCCACCCTCAGCGTCTGGGACCCCCGGGCAGAGGGTCACCAT
CTCTTGTTCTGGAAGCAGCTCCAATATCGGAAGTAATTATGTATA
CTGGTACCAGCAACTCCCAGGAACGGCCCCCAAACTCCTCATCTT
TAGGAATAATCAGCGGCCCTCAGGGGTCCCTGACCGATTCTCTGG
CTCCAAGTCTGGCACCTCAGCCTCCCTGGCCATCAGTGGGCTCCG
GTCCGATGATGAGGCTGATTATTACTGTGTAGCATGGGATGACAG
CCTGAGTGCTTGGGTGTTCGGCGGAGGGACCAAGCTCACCGTCCT
AGGTGAGGGTAAATCTTCCGGATCTGGTTCCGAATCCAAAGCTAG
CGAGGTGCAGCTGGTGCAGTCTGGGGGAGGCTTGGTACAGCCTGG
CAGGTCCCTGAGACTCTCCTGTGCAGCCTCTGGGTTCCCCTTTGA
TGATTATGCCATGCACTGGGTTCGGCAAGCTCCAGGGAAGGGCCT
GGAGTGGGTCTCAGGGATTAGTTGGAATAGTGGTAGTATAGGGTA
TGCGGACTCTGTGAAGGGCCGATTCACCATCTCCAGAGACAACGT
CAAGAACACACTATATCTGCAAATGAACAACCTGAGAGTCGAGGA
CACGGCCGTATATTATTGTGCGAAATTAGCCGGGGATATTGTGGT
GATTCCCCTCTGGGGCCCGGGCACCCTGGTCACCGTCTCCTCATA
A

### Anti-Huntington

4) Anti-Huntingtin scFv Antibody
DNA: Synthetic construct anti-huntingtin intrabody single chain Fv antibody gene, partial cds GenBank: EU490426.1 (726 bp) ncbi.nlm.nih.gov/nuccore/EU490426.1
Protein: anti-huntingtin intrabody single chain Fv antibody, partial [synthetic construct] GenBank: ACA53373.1 (242 aa) ncbi.nlm.nih.gov/protein/aca53373.1

### REFERENCES

Lecerf J M, Shirley T L, Zhu Q, et al. Human single-chain Fv intrabodies counteract in situ huntingtin aggregation in cellular models of Huntington's disease. Proc Natl Acad Sci USA. 2001; 98(8):4764-4769. pubmed.ncbi.nlm.nih.gov/11296304/
Wolfgang W J, Miller T W, Webster J M, et al. Suppression of Huntington's disease pathology in Drosophila by human single-chain Fv antibodies. Proc Natl Acad Sci USA. 2005; 102(32):11563-11568. pubmed.ncbi.nlm.nih.gov/16061794/
EU490426.1 Synthetic construct anti-huntingtin intrabody single chain Fv antibody gene, partial cds (726 bp)

(SEQ ID NO: 8)
ATGGCCCAGGTGCAGCTGCAGGAGTCGGGGGGAGGCTTGGTACAG
CCTGGGGGGTCCCTGAGACTCTCCTGTGCAGCCTCTGGATTCAC
CTTCAGTAGTTATAGCATGAGCTGGGTCCGCCAGGCTCCAGGCAA
GGGGCTGGAGTGGGTGGCAGTTATATCATATGATGGAAGCAATAA
ATACTACGCAGACTCCGTGAAGGGCCGATTCACCATCTCCAGAGA
CAATTCCAAGAACACGCTGTATCTTCAAATGAACAGCCTGAGAGC
CGAGGACACGGCCGTGTATTACTGTGCGAGAGATAGGTACTTCGA
TCTCTGGGGCCGTGGCACCCTGGTCACCGTCTCCTCAGGTGGAGG
CGGTTCAGGCGGAGGTGGCTCTGGCGGTGGCGGATCGCAGTCTGC
CCTGACTCAGCCTGCCTCCGTGTCTGGGTCTCCTGGACAGTCGAT
CACCATCTCCTGCACTGGAACCAGCAGTGACATTGGTGCTTATAA
CTATGTCTCCTGGTACCAGCAGTATCCAGGCAAGGCCCCCAAACT
CCTTATTTATGATGTCAGTAATCGGCCCTCAGGGATTTCTAATCG
CTTCTCTGGCTCCAAGTCTGGCGATACGGCCTCCCTGACCATCTC
TGGGCTCCAGGCTGAGGACGAGGCTGATTATTACTGCAGCTCATT
TGCGAACAGCGGCCCCTTATTCGGCGGAGGGACCAAGGTCACCGT
CCTAGGT

### Anti-Homer 1

### 5) Anti-Homer1 Nanobody

DNA: Synthetic construct HC20 pEYFPN1 anti-Homer1(Mouse) recombinant llama nanobody (Addgene plasmid #135220; http://n2t.net/addgene:135220; RRID:Addgene_135220)

### REFERENCE

Dong J X, Lee Y, Kirmiz M, Palacio S, Dumitras C, Moreno C M, Sando R, Santana L F, Sudhof T C, Gong B, Murray K D, Trimmer J S. A toolbox of nanobodies developed and validated for use as intrabodies and nanoscale immunolabels in mammalian brain neurons. Elife. 2019 Sep. 30; 8:e48750. doi: 10.7554/eLife.48750. PMID: 31566565; PMCID: PMC6785268.
HC20 pEYFPN1 anti-Homer1 nanobody
   >nanobody sequence trimmed between the 5′ and 3′ SFiI sites

(SEQ ID NO: 9)
ATGGCCGAGGTGCAGCTGGTGGAGTCTGGGGGAGGATTGGTGCAG
GCTGGGGGCTCTCTGAGACTCTCCTGTGCAGCCTCTGGACTTACT
GTCAATATGTGGTATACGGGCTGGTTCCACCAGGCTCCAGGGAAG
GAGCGTGAGTTTGTCGCAGGTATTAGTCAGAGTGGTGTTCGGACA
TACGTCGCAGACTCCGTGAAGGGCCGATTCACCATCTCCAGAGAC
AACGCCAAGAAGACGGTGTATCTGCAAATGAACAGCCTGAAACCT
GAGGACACGGGCGTTTATTACTGTGCAGGGGGTACTTGGGAGTCA
GAAGTACGGAGGGGCGTGCTGTCCTCCAGGGGCCAGGGGACCCAG
GTCACTGTCTCCTCA

### 2) Anti-Homer1 Nanobody

DNA: Synthetic construct HC87 pEYFPN1 anti-Homer1 (Mouse) recombinant llama nanobody (Addgene plasmid #135223; http://n2t.net/addgene:135223; RRID:Addgene_135223)

### REFERENCE

Dong J X, Lee Y, Kirmiz M, Palacio S, Dumitras C, Moreno C M, Sando R, Santana L F, Sudhof T C, Gong B, Murray K D, Trimmer J S. A toolbox of nanobodies developed and validated for use as intrabodies and nanoscale immunolabels in mammalian brain neurons. Elife. 2019 Sep. 30; 8:e48750. doi: 10.7554/eLife.48750. PMID: 31566565; PMCID: PMC6785268.
HC87 pEYFPN1 anti-Homer1 nanobody
   >nanobody sequence trimmed between the 5′ and 3′ SFiI sites

(SEQ ID NO: 10)
ATGGCCCAGGTGAAGCTGCAGGAGTCAGGGGGAGGATTGGTGCAG
GTTGGGGACTCTCTGAGACTCTCCTGTGCAGCCTCTGGACGCCCC
TTCAGTGATTATTACATGGCGTGGTTCCGCCAGGCTCCAGGGAAG
GAGCGTGAGTTTGTGGCAGTGATTGGCTGGAGTGGTCTGACCACA
TACTATGCAGACTCCGTGAAGGGCCGATTCACCATCTCCAGAGAC
AACGCCAAGAACACGGGATATCTGCAAATGAACAGCCTGAAACTT
GAGGACACGGCCGTTTATTACTGTGCAGCAGATCTCCGTCGCCGT
CTTAGTTTGACCGAATTTGACTACTGGGGCCAAGGGACCCAGGTC
ACCGTCTCCTCA

The targeting moiety can be any molecule that binds to the receptor which is differentially expressed on the target cell. Examples include transferrin (Tf), folate, other vitamins, EGF, insulin, Heregulin, RGD peptides or other polypeptides reactive to integrin receptors, antibodies or their fragments. Suitably the molecule is a transferrin receptor-targeting molecule that targets the transferrin receptor. An exemplary antibody fragment is a single chain Fv fragment of an antibody. The antibody or antibody fragment is one which will bind to a receptor on the surface of the target cell, and suitably to a receptor that is differentially expressed on the target cell. One exemplary antibody is an anti-TfR monoclonal antibody and a suitable antibody fragment is an scFv based on an anti-TfR monoclonal antibody (TfRscFv).

The targeting moiety (transferrin receptor-targeting molecule) is suitably mixed with a cationic liposome at room temperature and at a targeting moiety:liposome ratio in the range of about 1:0.001 to 1:500 (□g:nmole), preferably about 1:10 to about 1:50 (□g:nmole). Nucleic acid (suitably plasmid DNA) is mixed with the cationic liposome at room temperature and at an agent:lipid ratio in the range of about 1:0.1 to about 1:50 (□g:nmole), preferably about 1:10 to about 1:24 ( [symbol] g:nmole). In complexes, for example, in which the targeting moiety is transferrin and the nucleic acid is plasmid DNA, useful ratios of nucleic acid to liposome to targeting moiety typically are within the range of about 1 [symbol] g to 0.1-50 nmoles to 0.1-100 [symbol] g, preferably 1 [symbol] g to 5-24 nmoles to 6-36 [symbol] g, most preferably about 1 [symbol] g to 10 nmoles to 12.5 [symbol] g. If the targeting moiety is TfRscFv, useful ratios of targeting moiety to liposome typically are within the range of about 1:5 to 1:40 ( [symbol] g: [symbol] [symbol] g), preferably 1:30 ( [symbol] g: [symbol] [symbol] g), and the ratio of plasmid DNA to liposome typically is within the range of about 1:6 to 1:20 ( [symbol] g: [symbol] [symbol] g), preferably 1:10 ( [symbol] g: [symbol] [symbol] g).

A wide variety of cationic liposomes are useful in the preparation of the complexes described herein. Published PCT application WO 99/25320, incorporated herein by reference, describes the preparation of several cationic liposomes. Examples of desirable liposomes include those that comprise a mixture of dioleoyltrimethylammonium phosphate (DOTAP) and dioleoylphosphatidylethanolamine (DOPE) and/or cholesterol (chol), or a mixture of dimethyldioctadecylammonium bromide (DDAB) and DOPE and/or cholesterol, or a mixture of dimethyldioctadecylammonium bromide (DDAB) and DOPE, or a mixture of dioleoyltrimethylammonium phosphate (DOTAP) and dioleoylphosphatidylethanolamine (DOPE). The ratio of the lipids can be varied to optimize the efficiency of uptake of the therapeutic molecule for the specific target cell type. The liposome can comprise a mixture of one or more cationic lipids and one or more neutral or helper lipids. A desirable ratio of cationic lipid(s) to neutral or helper lipid(s) is about 1:(0.5-3), preferably 1:(1-2) (molar ratio).

### Exemplary Lipid Compositions Include:

| | | |
|---|---|---|
| LipA | DOTAP/DOPE | 1:1 molar ratio |
| LipB | DDAB/DOPE | 1:1 molar ratio |
| LipC | DDAB/DOPE | 1:2 molar ratio |
| LipD | DOTAP/Chol | 1:1 molar ratio |
| LipE | DDAB/Chol | 1:1 molar ratio |
| LipG | DOTAP/DOPE/Chol | 2:1:1 molar ratio |
| LipH | DDAB/DOPE/Chol | 2:1:1 molar ratio |

In embodiments, the administration is intravenous (IV), intratumoral (IT), intralesional (IL), aerosal, percutaneous, oral, endoscopic, topical, intramuscular (IM), intradermal (ID), intraocular (IO), intraperitoneal (IP), sublingual (SL), transdermal (TD), intranasal (IN), intracereberal (IC), intraorgan (e.g. intrahepatic), slow release implant, or subcutaneous administration, or via administration using an osmotic or mechanical pump.

In addition to targeting proteins associated with synucleinopathic disorders (including Parkinson's Disease), Alzheimer's Disease, Amyotrophic Lateral Sclerosis (ALS) disease and Huntington's disease, other embodiments include the targeting of other brain proteins involved in neurological disorders or conditions wherein the association of the antibody or antibody fragment with the protein in question would have therapeutic benefits. Other embodiments include the targeting a set of neuronal proteins with restricted expression in subcellular compartments associated with discrete signaling events crucial to mammalian brain neuron function and plasticity. These include postsynaptic scaffolding proteins that are present at partially overlapping sets of excitatory synapses.

### EXAMPLES

### Example 1

### Cationic Liposome Preparation

Liposomes A-H as described above (e.g., Lipid A (DOTAP:DOPE, 1:1 molar ratio) are prepared by an ethanol injection method modified from that described by Campbell, MJ (Biotechniques 1995 June; 18(6):1027-32). All lipids are solubilized in ethanol at concentrations of 20-100 mg/ml and mixed, injected into vortexing or rapidly stirring pure water of 50-65° C. with a Hamilton syringe. The solution is stirred slowly for a further 10-15 min until it returns to room temperature. For sterility purposes only, the solution is passed through a 0.22 μm pore Milex G V filter. No further processing, e.g. for size selection, is required or employed. Evaporation, sonication, milling and/or extrusion of the liposomes is specifically excluded from the disclosed methods. The final concentration of liposomes is 0.5 nmol/ul to 8 nmol/ul total lipids.

For Lipid A and Lipid B, a mixture of DOTAP:DOPE and DDAB/DOPE, respectively, both at a molar ratio of 1:1, is used with a preferred final concentration of 2 mM to 8 mM. The final size of the liposomes is between 20 nm and 100 nm, with a positive charge in the range of 10 to 50 mV as determined by dynamic light scattering using a Malvern Zetasizer ZS or Malvern Zetasizer Pro.

### Example 2

### Preparation of Tf-Targeted-Liposome-DNA Nanocomplexes for Delivery Across the Blood Brain Barrier for Transfection of Cells in the Brain for Treatment of Neurological Disorders.

The Tf-liposome-DNA complexes for use in delivering their payloads across the blood brain barrier and to neuronal cells are prepared by mixing Tf with the cationic liposome composition at defined ratios of Tf protein to liposome to DNA in the mixed complex in the ranges described throughout. The ratios used are as follows: the DNA to liposome to ligand ratio is 1 μg to 0.1-50 nmole to 0.1-100 μg, preferably 1 μg to 5-24 nmole to 6-36 μg and most preferably 1 μg to 10 nmoles to 12.5 μg.

The DNA used is a plasmid carrying the DNA sequence encoding a gene. This plasmid also contains enhancer sequences upstream of the promoter and the gene is under the control of a high expression promoter. The gene encodes a protein which is a single chain antibody fragment targeted to a gene associated with a neurological disease, including, but not limited to, synucleinopathic disorders (including Parkinson's Disease), Alzheimer's Disease, Amyotrophic Lateral Sclerosis (ALS) disease and Huntington's disease.

Cationic liposomes consisting of dioleoyl trimethylammonium propane (DOTAP) and dioleoyl phosphatidylethanolamine (DOPE) (Avanti Polar Lipids, Inc., Alabaster, AL) were prepared as above and mixed with any water (e.g., DI water) if required to give a desired volume and gently inverted at least 10 times to mix. The final concentration of liposomes was 2 nmol/ul (2 mM). For in vitro studies, Holo-transferrin (Tf, iron-saturated, Sigma) was dissolved in pure water at 5 mg/ml. To prepare the Tf-liposome-DNA complex, 12 nmol of liposomes were added to 18 ug Tf in 100 μl serum-free EMEM, mixed by gentle inversion for about 5-10 seconds or for larger volumes rotated at 20-30 RPM for 1-2 minutes and incubated for 5-15 min at room temperature with frequent rocking. This solution was then mixed with 1.2 μg plasmid DNA in 100 μl serum-free media (e.g. EMEM) mixed by gentle inversion for about 5-10 seconds or for larger volumes rotated at 20-30 RPM, and incubated for 15-30 minutes at room temperature with frequent rocking. The prepared Tf-liposome (designated LipT(A)-DNA) complex was used for in vitro cell transfection freshly within 1 hour of preparation, although it was found to be stable for at least 24 hours with the same transfection efficiencies. Agarose gel electrophoresis was employed to assess the DNA retardation by LipT(A). Greater than 90% of the DNA was found to be complexed to the liposome, and actually encapsulated within the liposome. The size (number average) of the final complex prepared as above is between about 50 to 200 nm with a zeta potential of between about 20 to 50 mV as determined by dynamic light scattering using a Malvern Zetasizer ZS or Malvern Zetasizer Pro.

For in vivo studies, the liposome and transferrin (in water) at the amounts given above, were mixed by gentle inversion for about 5-10 seconds or for larger volumes rotated at 20-30 RPM for 1-2 minutes and incubated for 5-15 min at room temperature with frequent rocking. This solution is then mixed with DNA (in HEPES buffer pH=7.4, in water, or in 10 mM Tris-1 mM EDTA Buffer pH=7.5) by gentle inversion for about 5-10 seconds or for larger volumes rotated at 20-30 RPM for 1-2 minutes and incubated for 5-30 min at room temperature with frequent rocking. A 50% dextrose solution was added to reach a final concentration of 5% dextrose or a 50-70% sucrose solution was added to reach a final 10% sucrose concentration, mixed by inversion at least 10 times and checked for signs of precipitation (the presence of particulate matter or cloudiness). The size (number average) of the final complex prepared as above is between about 50 to 200 nm with a zeta potential of between about 20 to 50 mV as determined by dynamic light scattering using a Malvern Zetasizer ZS or Malvern Zetasizer Pro. The LipT(A)-DNA complex was found to be relatively stable for up to 24 hours at 4° C. in the dark, without substantial loss of transfection efficiency.

No further manipulation (e.g. milling, extrusion, filtration, or any method of separation) is required or takes place.

When unliganded complex (LipB-DNA) was used as a control, a mixture of DDAB/DOPE (Avanti Polar Lipids, Inc., Alabaster, AL), at a molar ratio of 1:1, was used with a preferred final concentration of 2 mM to 8 mM. To make this nanocomplex, the cationic liposomes (2 mM) were prepared as above and mixed with any water (e.g., DI water), if required to give a desired volume and gently inverted at least 10 times to mix. This solution was then mixed with 1.2 μg plasmid DNA in 100 μl serum-free media (e.g.EMEM) mixed by gentle inversion for about 5-10 seconds or for larger volumes rotated at 20-30 RPM, and incubated for 15-30 minutes at room temperature with frequent rocking. The prepared liposome-DNA (designated LipT(B)-DNA) complex was used for in vitro cell transfection freshly within 1 hour of preparation, although it was found to be stable for at least 24 hours with the same transfection efficiencies. The size (number average) of the final complex prepared as above is between about 50 to 200 nm with a zeta potential of between about 20 to 50 mV as determined by dynamic light scattering using a Malvern Zetasizer ZS or Malvern Zetasizer Pro.

The nanocomplex can be used immediately after preparation or lyophilized to dryness and stored at −20 to 8° C. prior to use. If lyophilized, the nanocomplex is reconstituted by the addition of an amount of sterile, endotoxin free water equivalent to the original volume prior to lyophilization. After swirling for 30 sec to 1 min to dissolve, the vial may be sonicated in a bath sonicator (for example the Elma Elmasonic 30P ultrasonic water bath) at 37 kHz, 100% power for 1 to 10 minutes at 31-34° C. The vial is swirled gently every 30-60 sec during the sonication. Alternatively, the reconstituted solution can be rotated at 30 RPM for 30 sec to 10 minutes at room temperature.

### Example 3

### Systemic Delivery of Tf-Targeted Liposome Nanocomplex Across the BBB to Neuronal Cells in Normal Rats.

Transferrin (Tf)-complexed liposome-DNA (Tf-LipA+cDNA) was made as above in Example 2. Non-targeted cationic liposome-DNA (LipB+cDNA) was prepared as described in Example 2. To assess the feasibility of using the TfR-targeted nanocomplex as a vehicle for specific neuronal targets, experiments were performed in vivo by systemically (intravenous) delivering transferrin (Tf)-complexed liposomes (Tf-LipA) (FIG. 4, panels A1, A2) and non-targeted cationic liposomes (LipB) (FIG. 4, panel B) into normal rats. Previous studies have shown that TfRs are abundant at both BBB endothelium and neurons. Thus, these experiments assessed the ability of the TfR-targeted liposomes of this application to cross the BBB after systemic administration and also target post-mitotic neuronal cells. A single bolus (1 ml) of either Tf-LipA or LipB, both of which encapsulated EGFP cDNA (100 ug cDNA/ml) was intravenously injected via a catheterized jugular vein in anesthetized rats. 24-36 hours post-injection, the animals were sacrificed and coronal 40 um brain sections analyzed via fluorescence microscopy. As expected, non-targeted liposomes-DNA produced no detectable EGFP(+) cells in the brain (FIG. 4, panel B). In contrast, Tf-LipA+cDNA injections yielded widespread EGFP expression (mainly in neuronal cells) in several brain areas (cortex and hippocampus) which indicate that the TfR-targeted nanocomplex does cross the BBB and does deliver the genetic payload to adult neurons. Parvalbumin immunostaining confirmed that EGFP(+) neurons belong to distinct populations. Higher DNA doses enhances efficiency for long-lasting expression. This data support the use of the targeted nanoliposome complexes for gene delivery to post-mitotic neurons in the brain.

### Example 4

### Preparation of TfRscFv-Targeted-Liposome-DNA Nanocomplexes for Delivery Across the Blood Brain Barrier for Transfection of Cells in the Brain for Treatment of Neurological Disorders

It has been determined that a simple mixing of the TfRscFv and the cationic liposome (which does not contain any lipid with a reducible group such as Maleimide DOPE or any reducible group), instead of chemical conjugation, results in formation of an immunologically active complex, wherein the targeting moiety is bound directly to the liposome without the use of a linker or chemical-conjugation molecule (see e.g., U.S. Published Patent Application No. 2003/0044407, the disclosure of which, including the compositions and methods disclosed therein, is incorporated by reference herein), that still efficiently crosses the blood-brain barrier, binds to and transfects neuronal cells.

In the examples given below in this Example and in Examples 5, 6 and 7, the cationic liposomes consisting of dioleoyl trimethylammonium propane (DOTAP) and dioleoyl phosphatidylethanolamine (DOPE) (Avanti Polar Lipids, Inc., Alabaster, AL) are prepared as in Example 1. The transferrin receptor-targeting molecule is TfRscFv or any antibody or antibody fragment (including any scfv, Fab′ or Mab). The targeted immunoliposome complexes are prepared by mixing the TfRscFv or any antibody or antibody fragment as above with any of the liposome compositions given above at defined ratios of protein to liposome and DNA in the mixed complex. Preferably, the cationic liposomes, prepared as above, consist of dioleoyl trimethylammonium propane (DOTAP) and dioleoyl phosphatidylethanolamine (DOPE) (Avanti Polar Lipids, Inc., Alabaster, AL) at a final concentration of 2 to 8 mM. The preparation of the complexes is in accordance with the following general procedure.

The appropriate amount of 2 mM liposome is mixed with any water (e.g., DI water) required to give a desired volume and gently inverted 10 times to mix, or for larger volumes rotated at 20-30 RPM for 1-2 minutes. To the liposome-water mixture, the appropriate amount of TfRscFv (or other targeting antibody or antibody fragment) is added to give the desired ratio and mixed by gentle inversion for about 5-10 seconds or for larger volumes rotated at 20-30 RPM for 1 minute. This mixture is kept at room temperature for 10 to about 30 minutes (again inverted gently for 5-10 seconds after approximately 5 minutes). At the same time, the appropriate amount of nucleic acid, preferably one or more plasmid DNAs carrying a gene or genes encoding an intrabody antibody or an antibody fragment is mixed by inversion for 5-10 seconds, or for larger volumes rotated at 20-30 RPM for 1-2 minutes, with any water required to give a desired volume. The gene or genes can encode a complete antibody (IgG), an Fab, and F(ab′)₂, a mono- or bi-specific Fab₂, Tri-specific Fab₃, A monovalent IgG, a bi- or tri-specific diabody, an scFv-Fc fragment, a minibody, an IgNAR a V-NAR, a hclIgG or a VhH fragment. Other nucleic acids can also be used in place of plasmid DNA including, but not limited to, siRNA, miRNA, mRNA. The DNA solution is quickly added to the targeted-liposome solution and the mixture is inverted for 5-10 seconds or for larger volumes rotated at 20-30 RPM for 1-2 minutes. The final mixture is kept at room temperature for 10 to about 30 minutes, gently inverting again for 5-10 seconds after approximately 5 minutes. Typically, for use in an in vitro assay, it is desirable that the concentration of DNA is in the range of about 0.01 μg to about 10 g per well; for in vivo use in animals, it is desirable to provide about 5 μg to about 100 μg of DNA per injection. In humans it is desirable to provide 0.1 to 10 mg, preferably 0.6 to 7.2 mg of DNA/injection.

For in vitro use, the targeted cationic liposome nanocomplex carrying plasmid DNA, e.g. a nucleic acid encoding a single chain FV antibody fragment (scFV), is further diluted with serum-free media (SFM) for use in transfection. For use in vivo 50% dextrose or 50-70% sucrose is added to the targeted cationic liposome nanocomplex carrying plasmid DNA, e.g. a nucleic acid encoding a single chain FV antibody fragment (scFV), to a final concentration of 1-20% (V:V), suitably 5-20% (V:V) and mixed by gentle inversion for about 1 second to about 2 minutes or for larger volumes rotated at 20-30 RPM for 1-2 minutes.

No further manipulation (e.g. milling, extrusion, filtration, or any method of separation) is required or takes place. The size (number average) of the final complex prepared by these methods is between about 10 to 800 nm, suitably about 50 to 400 nm, most suitably about 25 to 200 nm with a zeta potential of between about 1 and 100 mV, more suitably 10 to 60 mV and most suitably 20 to 50 mV as determined by dynamic light scattering using a Malvern Zetasizer ZS or Malvern Zetasizer Pro.

Useful ratios of targeting moiety to liposome typically are within the range of about 1:5 to 1:40 ( [symbol] g: [symbol] [symbol] g), preferably 1:30 ( [symbol] g: [symbol] [symbol] g), and the ratio of plasmid DNA to liposome typically is within the range of about 1:6 to 1:20 ( [symbol] g: [symbol] [symbol] g), preferably 1:10 ( [symbol] g: [symbol] [symbol] g). A specific example at a suitable ratio of 1:30 (TfRscFv:liposome, w:w) and 1:14 (μg DNA:n mole total Lipid) is as follows. For 40 μg of DNA in a final volume of 800 μl, mix 183 μl water with 280 μl of 2 mM liposome solution. Add 34 μl of TfRscFv (with a concentration of 0.4 μg/ml). Mix 183 μl water with 40 μl of 1 μg/1 μl DNA. Add 80 μl of 50% Dextrose as the last step.

The nanocomplex can be used immediately after preparation or lyophilized to dryness and stored at −20 to 8° C. prior to use. If lyophilized, the nanocomplex is reconstituted by the addition of an amount of sterile, endotoxin free water equivalent to the original volume prior to lyophilization. After swirling for 30 sec to 1 min to dissolve, the vial may be sonicated in a bath sonicator (for example the Elma Elmasonic 30P ultrasonic water bath) at 37 kHz, 100% power for 1 to 10 minutes at 31-34° C. The vial is swirled gently every 30-60 sec during the sonication. Alternatively, the reconstituted solution can be rotated at 30 RPM for 30 sec to 10 minutes at room temperature.

The size (number average) of the final complex prepared by the methods is between about 10 to 800 nm, suitably about 50 to 400 nm, most suitably about 25 to 200 nm with a zeta potential of between about 1 and 100 mV, more suitably 10 to 60 mV and most suitably 20 to 50 mV as determined by dynamic light scattering using a Malvern Zetasizer ZS or Malvern Zetasizer Pro. This size is small enough to efficiently pass through the blood-brain barrier by receptor-mediated transcytosis and reach neuronal cells for uptake.

### Example 5

### Use of TfR-Targeted Cationic Liposome Complexes for Treatment of Neurological Diseases

The present invention provides methods for treating neurologic diseases in humans comprising administering to the individual a transferrin receptor targeted cationic liposome complex comprising a nucleic acid molecule encoding a single chain FV antibody fragment (scFV) against a protein associated with a neurologic disease or disorder, for example, a nucleic acid encoding a single chain FV antibody fragment (scFV) against alpha-synuclein (α-Syn) for the treatment of Parkinson's disease. The complexes comprising various targeting moieties directly complexed/associated with, but not chemically conjugated to, the surface of the liposomes is prepared essentially as described above in Examples 2 and 4 at defined ratios of targeting moiety to liposome and DNA to n moles (or Dg) total lipid in the mixed complex in the ranges described throughout.

A specific example for use in humans at a suitable ratio of 1:30 (TfRscFv:liposome, w:w) and 1:14 (g DNA:n mole total Lipid) is as follows. For 0.6 mg of DNA in a final volume of 7 ml, To 4.2 ml of 2 mM liposome solution is added 0.382 of TfRscFv (with a concentration of 0.514 mg/ml). After mixing and holding at room temperature as per Example 4, 1.2 ml of 0.5 mg/ml DNA is added. After mixing and holding at room temperature as per Example 4, 1.22 mL of 57.4% sucrose is added as the last step.

The complex can be used after being freshly prepared. Alternatively, the complex can be lyophilized and stored at Room Temperature, 2-8° C., −20° C., or −70 to −80° C. for at least for 7 years, until use. If lyophilized, the complex of this invention is first reconstituted with sterile, endotoxin free water equivalent to the original volume prior to lyophilization as described in Example 1. After swirling for 30 sec to 1 min to dissolve, the vial is sonicated in a bath sonicator (for example the Elma Elmasonic 30P ultrasonic water bath) at 37 kHz, 100% power for about 6 to 10 minutes at 31-34° C. The vial is swirled gently every 30-60 sec during the sonication. Alternatively, the reconstituted solution can be rotated at 30 RPM for 30 sec to 10 minutes at room temperature. The freshly prepared or reconstituted nanocomplex is administered as an iv infusion in 5% dextrose solution.

The Tf-Lip-DNA complex is prepared according to the in vivo formulation described previously in Example 2. For a typical preparation with optimized in vivo formulation, 250 ul of Tf (5 mg/ml, iron-saturated holo-transferrin; Sigma, St. Louis, MO) plus 750 ul of water are mixed in a polypropylene to which is added 500 ul of Lip (2 mM total lipids). The components are mixed by gentle inversion10 times and held for 5-15 min at room temperature with frequent rocking, or for larger volumes rotated at 20-30 RPM for 1-2 minutes. One hundred micrograms of plasmid DNA in 1.2 ml of 20 mM HEPES buffer, pH 7.4 (or sterile endotoxin free water), is added to the tube, mixed immediately and thoroughly by gentle inversion 10 times, or for larger volumes rotated at 20-30 RPM for 1-2 minutes, and held for 10-20 min at room temperature with frequent gentle inversion. Three hundred microliters of 50% dextrose solution, or 430 microliters of 70% sucrose is then added to the tube, mixed immediately and thoroughly by gentle inversion 10 times, or for larger volumes rotated at 20-30 RPM for 1-2 minutes, and held at room temperature for 10-15 minutes. The final DNA:lipid:Tf ratio is 1:10:12.5 (mg/nmol/mg). The HEPES buffer can be replaced by water. The complex can be used after being freshly prepared. Alternatively, the complex can be lyophilized and stored at Room Temperature, 2-8° C., −20° C., or −70 to −80° C. until use. If lyophilized, the complex of this invention is first reconstituted in pure water as described in Example 2.

### Use Against Synucleinopathic Disorders (Including Parkinson's Disease)

If the disease to be treated is a synucleinopathic disorder, in one embodiment Parkinson's disease, the nucleic acid carried by the plasmid encapsulated in the TfR targeted nanocomplex encodes a scFv antibody (an “intrabody”) against [symbol] Syn monomers to inhibit their aggregation. An intrabody against [symbol] Syn can both reduce production of [symbol] Syn in the cells of origin and “immunize” recipient cells against incoming [symbol] Syn originating in another cell. In other embodiments the TfR targeted nanocomplex encoding a scFv antibody (an “intrabody”) against alpha-Syn monomers can be used to treat patients with dementia with Lewy bodies, or multiple system atrophy.

### Use Against Alzheimer's Disease

If the disease to be treated is Alzheimer's Disease, the nucleic acid carried by the plasmid encapsulated in the TfR targeted nanocomplex encodes a scFv antibody (an “intrabody”) against the Aβ peptide resulting from the cleavage of the amyloid precursor protein. This peptide is prone to self-aggregation, forming neurotoxic species, and the therapy described in this application is aimed at reversing the formation of these aggregates.

### Use Against Amyotrophic Lateral Sclerosis (ALS) Disease

If the disease to be treated is Amyotrophic Lateral Sclerosis (ALS) Disease, the nucleic acid carried by the plasmid encapsulated in the TfR targeted nanocomplex encodes a scFv against the Cu/Zn superoxide dismutase (SOD1). Misfolding and aggregation of mtSOD1 is a consistent feature of the pathology of mtSOD1-induced ALS in patients. and has been proposed to underlie the basis for motor neuron degeneration. Aggregates of mtSOD1 could cause toxicity by e,g, sequestering SOD1-binding proteins that are critical to the viability of motor neurons. The therapy described in this application is aimed at preventing and/or reversing the formation of the misfolding and/or aggregation.

### Use Against Huntington's Disease

If the disease to be treated is (HD) Disease, the nucleic acid carried by the plasmid encapsulated in the TfR targeted nanocomplex encodes a scFv antibody (an “intrabody”) against the mutant form of the huntingtin protein (mtHTT) Huntington's Disease (HD) is characterized by abnormal folding and proteolytic cleavage of this mHTT to N-terminal fragments, leading to formation of aggregates and neuronal and neuropil inclusion bodies in the brain. The therapy described in this application is aimed at preventing and/or reversing the formation of these aggregates.

The size of the final complexes prepared by the methods described above in Examples 2 and 4 carrying the plasmids encoding the scFv intrabodies described in this specification are between about 50 and 500 (nm) with a positive zeta potential as determined by dynamic light scattering using a Malvern Zetasizer® Nano-ZS or a Malvern Zetasizer Pro. This size is small enough to efficiently pass through the capillary bed and reach the target neuronal cells in the CNS to deliver the plasmid payload into the neuronal cells.

The complexes, prepared as above, are suitably used as a therapeutic agent. The anti TfR-targeted cationic liposome nanocomplex, prepared as above, is injected into humans diagnosed with a neurologic disorder or who are at risk of developing a neurologic disorder through genetic means. When Tf is used as the targeting moiety, the complex used to treat the subject (patient) is suitably made at ratios of 1:10:12.5 (mg/nmol/mg) (DNA:lipid:Tf) with 5-20% Dextrose or Sucrose as an excipient. When TfRscFv is the targeting moiety of the nanocomplex, the complex used to treat the subject (patient) is suitably made at ratios of 0.33 ug:10 ug:1 ug (TfRscFv:Lip:DNA), with 5-20% Dextrose or Sucrose as an excipient.

The total amount of complexed DNA administered to the patient per injection is 0.01 to 12 mg/injection. A suitable amount is 0.164 mg/kg/injection which is 7-12 mg DNA/Injection, based upon the weight of the subject. The prepared complex is injected either into a 250 ml 5% Dextrose bag for i.v. infusion, or injected as a bolus, by intravenous (IV), intratumoral (IT), intralesional (IL), aerosal, percutaneous, oral, endoscopic, topical, intramuscular (IM), intradermal (ID),transdermal (TD), intraocular (IO), intraperitoneal (IP), intranasal (IN), intracereberal (IC), intraorgan (e.g. intrahepatic), slow release implant, or subcutaneous administration, or via administration via an osmotic or mechanical pump. The therapeutic TfR targeted nanocomplex is administered on a schedule appropriate for the specific neurologic disease. In some embodiments, administration is three times/week, or twice weekly, or once/week every week for a time ranging from months to years. In another embodiment, the administration is three times/week, or twice weekly, or once/week every two to 12 weeks for a time ranging from months to years.

### Example 6

Systemic Delivery of TfR-Targeted Liposome Nanocomplex Carrying Plasmid Encoding an Intrabody into Neuronal Cells of Normal Mice.

The feasibility of using the systemically administered TfRscFv-targeted nanocomplex as a vehicle for delivering genes encoding intrabodies into specific neuronal targets is assessed by determining the ability of the TfRscFv-targeted liposomes of this application to cross the BBB after systemic administration and also target post-mitotic neuronal cells. Experiments are performed in vivo by systemically (intravenously) delivering TfRscFv-targeted cationic liposomes into normal mice. TfRscFv-LipA (scL) encapsulating YFP-tagged anti-Homer1 nanobody (nAb) cDNA, prepared as described in Example 4, is intravenously injected via the tail vein into normal 6-15 week old Female or male mice. The amount of DNA encoding the intrabody administered via nanocomplex is 5 μg to about 100 μg of DNA per injection, preferably 30 ug. The mice are injected up to three times within a 24 hour period. At 6 to 120 hours, suitably 12 to 96 hours, most suitably 24-72 hours post-injection (or after the last injection when multiple injections were given, the animals are humanely euthanized and coronal 40 □m brain sections analyzed via fluorescence microscopy. TfRscFv-LipA-DNA nanocomplex injection(s)-YFP expression in neuronal cells in several brain areas, including hippocampus are measured following TfRscFv-LipA-DNA nanocomplex injection(s). Accumulation at synapses, which indicates that the TfRscFv-targeted nanocomplex does cross the BBB and does deliver the genetic payload to adult neurons are expected in staining results. Homer1 immunostaining suitably co-localizes with YFP expression indicating the affinity of nAb to target the postsynaptic scaffolding proteins Homer1) that is present at partially overlapping sets of excitatory synapses. This protein is also associated with Fragile X Syndrome (FXS) and Deafness Autosomal Dominant 68 (DFNA68).

### Example 7

### Preparation of Tf Receptor-Targeted-Liposome-DNA Nanocomplexes for Delivery of an Antibody or Antibody Fragment Across the Blood Brain Barrier for Transfection of Cells in the Brain for Treatment of Neurological Disorders.

The cationic liposomes are prepared as described above in Example 1. The Tf receptor targeted-liposome complexes for use in delivering antibody or antibody fragments (e.g. a complete antibody (IgG), an Fab, an F(ab′)2, a mono- or bi-specific Fab₂, Tri-specific Fab₃, A monovalent IgG, a bi- or tri-specific diabody, an scFv-Fc fragment, a minibody, an IgNAR a V-NAR, a hclIgG or a VhH fragment) across the blood brain barrier and to neuronal cells are prepared essentially as described in Examples 2 and 4 by mixing the targeting moiety, which can be Tf or TfRscFv, with the cationic liposome composition at defined ratios of targeting moiety to liposome to DNA in the mixed complex in the ranges described throughout. The targeting moiety (TfR-targeting molecule) is suitably mixed with a cationic liposome at room temperature and at a targeting moiety:liposome ratio in the range of about 1:0.001 to 1:500 (ug:nmole), preferably about 1:10 to about 1:50 (ug:nmole). The antibody/antibody fragment payload (e.g., IgG, scFv, Fab′ or Mab) is diluted with 10 mM Tris-buffer, pH 8.0 to pH 9.5 to a concentration of 10 mg/ml to 1 mg/ml and mixed with the cationic liposome at room temperature and at an antibody:lipid ratio in the range of about 1:0.5 to 1:4 (ug:ug), preferably 1:1.5 (ug:ug).

Preferably, the cationic liposomes, prepared as above, consist of dioleoyl trimethylammonium propane (DOTAP) and dioleoyl phosphatidylethanolamine (DOPE) (Avanti Polar Lipids, Inc., Alabaster, AL) at a final concentration of 2 to 8 mM. The preparation of the complexes is in accordance with the following general procedure where an exemplary ratio is 20 ug antibody/antibody fragment:30 ug Liposome:1 ug TfR targeting moiety. When prepared as described, the size of the nanocomplex is expected to be in the range of 98 nm to 300 nm (number average) based upon the amount of antibody/antibody fragment encapsulated by dynamic laser light scattering using a Malvern Nanosizer.

One embodiment for preparation with a mixture of mouse IgG and Fluorescent Antibody employs TfRscFv as the targeting moiety. In this embodiment for use in vitro, the complex is prepared as follows:

Complexes were Made with Mouse IgG “Cold” and Fluorescent “Hot” Antibody at Ratio:

[126] 98% “cold—2% “hot”, 95% “cold—5% “hot”, 90% “cold—10% “hot”, 80% “cold”—20% “hot”

### Materials:

Mouse IgG whole molecule −010-0102, (Rockland antibodies and assays, Limerick, PA), 10 mg/ml, “cold”.

Mitochondria Antibody (SPM198) (DyLight 550), NBP2-34745R, 0.55 mg/ml, (Novus Biologicals, cat #NBP2-34745R, Litleton, CO) —“hot” Ab

### Methods

TfRscFv-Lip-IgG Complex Preparation for Transfection (80% “Cold”-20%“Hot” Antibodies) RATIO: 20 ug IgG:30 ug Lip:1 ugTfRscFv

| | |
|---|---|
| Lip, 2 mM | 21 ul (30 ug) |
| LAL water | 27 ul |
| TfRscFv GP437, 0.514 mg/ml | 2 ul |

### Mixture of Antibodies:

“cold”−16 ug+“hot”−4 ug−450 ul in Serum free media
Dilute “cold” IgG (10 mg/ml) to lmg/ml with 10 mM Tris-buffer, pH 8.0.

Mix 2 mM Liposome (Lip) 21 ul (30 ug) with 27 ul of LAL water, add TfRscFv 2 ul (1 ug), mix by gentle inversion 10 times, hold at RT for 10 min. Total volume=50 ul TfRscFv-Lip

Take 16 ul (16 ug) of “cold” IgG (1 mg/ml) a add to 426.7 ul Serum Free media (SFM). Add 7.27 ul (4 ug) of “hot” IgG, mix by gentle inversion 10 times, hold at RT for 10 min. Total volume=450 ul.

Add antibody mixture to the 50 ul of TfRscFv-Lip (scL), mix by gentle inversion 10 times, hold at RT for 10 min. Total=20 ug antibody in 500 ul of TfRscFv-Lip-Ab complex.

No further filtration, centrifugation or extrusion was used after complex preparation. The complex can be used for transfection. Concentration IgG is 40 ug/ml

In this embodiment for use in vivo, the complex is prepared as follows:

### Materials:

Mouse IgG whole molecule −010-0102, (Rockland antibodies and assays, Limerick, PA), 10 mg/ml, “cold”.

Mitochondria Antibody (SPM198) (DyLight 550), NBP2-34745R, 0.55 mg/ml, (Novus Biologicals, cat #NBP2-34745R, Litleton, CO) —“hot” Ab

### Methods

RATIO: 20 ug IgG:30 ug Lip:1 ugTfRscFv

### Per Mouse:

| | |
|---|---|
| Lip, 2 mM | 21 ul (30 ug) |
| LAL water | 27 ul |
| TfRscFv GP437, 0.514 mg/ml | 2 ul |
| “cold” IgG, 1 mg/ml | 16 ul |
| “hot” Ab | 7.27 ul |
| Dextrose | 66 ul |
| SF media | 520.7 ul |
| Total: 20 ug Antibody in 660 ul | 660 ul |

Inject 20 ug IgG in 660 ul per mouse

The TfRscFv-Lip-Ab complex is made by mixing the components as described for in vitro use, with the exception that 66 ul of 50% Dextrose is added at the end (final conc 5%), mixed by gentle inversion 10 times, and held at RT for 10 min.

No further filtration, centrifugation or extrusion was used after complex preparation. The size of the nanocomplex was found to be approximately 130 nm (number average) by dynamic light scattering using a Malvern Nanosizer. Using a Vivaspin 500, 300000 MWCO PES filtration system (Sartozius Stedian Biotech, Goettingen, Germany) it was found that 92.3% of the antibody was encapsulated in the nanocomplex.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A method of treating a neurologic disorder in a patient, comprising:
providing a transferrin receptor-targeted cationic liposomal complex, comprising:
a cationic liposome;
a transferrin receptor-targeting molecule that is complexed with the cationic liposome, but is not chemically conjugated to the cationic liposome, and
wherein the transferrin receptor-targeting molecule does not comprise a lipid tag; and
a nucleic acid encoding an intrabody against a protein associated with the neurologic disorder; and
administering the transferrin receptor-targeted cationic liposomal complex to the patient to treat the disorder.

2. The method of claim 1, wherein the intrabody is a single chain FV antibody fragment (scFv) or a single domain antibody fragment.

3. (canceled)

4. The method of claim 1, wherein the transferrin receptor-targeting moiety is a single chain Fv antibody fragment.

5. The method of claim 4, wherein the single chain Fv antibody fragment is an anti-transferrin receptor single chain Fv (TfRscFv).

6. The method of claim 1, wherein the transferrin receptor-targeting moiety is a single domain antibody fragment.

7. The method of claim 1, wherein the transferrin receptor-targeting moiety is a transferrin molecule.

8. The method of claim 1, wherein the targeting moiety-targeted cationic liposomal complex is administered via intravenous (IV), intratumoral (IT), intralesional (IL), aerosal, percutaneous, oral, endoscopic, topical, intramuscular (IM), intradermal (ID), intraocular (IO), intraperitoneal (IP), sublingual (SL), transdermal (TD), intranasal (IN), intracereberal (IC), intraorgan (e.g. intrahepatic), slow release implant, subcutaneous administration, or via administration using an osmotic or mechanical pump.

9. The method of claim 1, wherein the transferrin receptor-targeted cationic liposomal complex crosses the blood brain barrier, and the nucleic acid encoding the intrabody is expressed in a neuronal cell.

10. The method of claim 1, wherein the neurologic disorder is a synucleinopathic disorder selected from the group consisting of Parkinson's Disease, dementia with Lewy bodies, Huntington's Disease, Alzheimer's, Amyotrophic Lateral Sclerosis (ALS), and multiple system atrophy.

11. (canceled)

12. (canceled)

13. The method of claim 1, wherein the cationic liposome comprises a mixture of dioleoyltrimethylammonium phosphate (DOTAP) and dioleoylphosphatidylethanolamine (DOPE) and/or cholesterol (chol), or a mixture of dimethyldioctadecylammonium bromide (DDAB) and DOPE and/or cholesterol, or a mixture of dimethyldioctadecylammonium bromide (DDAB) and DOPE, or a mixture of dioleoyltrimethylammonium phosphate (DOTAP) and dioleoylphosphatidylethanolamine (DOPE).

14. A method of treating a neurologic disorder in a patient, comprising:
providing a transferrin receptor-targeted cationic liposomal complex, comprising:
a cationic liposome;
a transferrin receptor-targeting molecule that is complexed with the cationic liposome, but is not chemically conjugated to the cationic liposome, and
wherein the transferrin receptor-targeting molecule does not comprise a lipid tag; and
an intrabody against a protein associated with the neurologic disorder; and
administering the transferrin receptor-targeted cationic liposomal complex to the patient to treat the disorder.

15. The method of claim 14, wherein the intrabody is a single domain antibody fragment, a single chain FV antibody fragment (scFv), an antibody (IgG), an Fab, and F(ab′)2, a mono- or bi-specific Fab2, a Tri-specific Fab3, a monovalent IgG, a bi- or tri-specific diabody, a minibody, an IgNAR a V-NAR, a hclIgG or a VhH fragment.

16. (canceled)

17. The method of claim 14, wherein the transferrin receptor-targeting moiety is a single chain Fv antibody fragment.

18. The method of claim 17, wherein the single chain Fv antibody fragment is an anti-transferrin receptor single chain Fv (TfRscFv).

19. The method of claim 14, wherein the transferrin receptor-targeting moiety is a single domain antibody fragment.

20. The method of claim 14, wherein the transferrin receptor-targeting moiety is a transferrin molecule.

21. The method of claim 14, wherein the targeting moiety-targeted cationic liposomal complex is administered via intravenous (IV), intratumoral (IT), intralesional (IL), aerosal, percutaneous, oral, endoscopic, topical, intramuscular (IM), intradermal (ID), intraocular (IO), intraperitoneal (IP), sublingual (SL), transdermal (TD), intranasal (IN), intracereberal (IC), intraorgan (e.g. intrahepatic), slow release implant, subcutaneous administration, or via administration using an osmotic or mechanical pump.

22. The method of claim 14, wherein the transferrin receptor-targeted cationic liposomal complex crosses the blood brain barrier, and the intrabody is delivered to a neuronal cell.

23. The method of claim 14, wherein the neurologic disorder is a synucleinopathic disorder selected from the group consisting of Parkinson's Disease, dementia with Lewy bodies, Huntington's Disease, Alzheimer's, Amyotrophic Lateral Sclerosis (ALS), and multiple system atrophy.

24. (canceled)

25. (canceled)

26. The method of claim 14, wherein the cationic liposome comprises a mixture of dioleoyltrimethylammonium phosphate (DOTAP) and dioleoylphosphatidylethanolamine (DOPE) and/or cholesterol (chol), or a mixture of dimethyldioctadecylammonium bromide (DDAB) and DOPE and/or cholesterol, or a mixture of dimethyldioctadecylammonium bromide (DDAB) and DOPE, or a mixture of dioleoyltrimethylammonium phosphate (DOTAP) and dioleoylphosphatidylethanolamine (DOPE).
